(19) United States
(12) Patent Application Publication
(10) Pub. No.: US 2024/0212791 A1
(43) Pub. Date: Jun. 27, 2024

(54) CONTEXT-DEPENDENT BASE CALLING

(71) Applicant: Illumina, Inc., San Diego, CA (US)

(72) Inventors: Gery Vessere, San Francisco, CA (US); David Olmstead Bracher, San Diego, CA (US); Aathavan Karunakaran, Berkeley, CA (US)

(21) Appl. No.: 18/391,487

(22) Filed: Dec. 20, 2023

Related U.S. Application Data
(60) Provisional application No. 63/476,428, filed on Dec. 21, 2022.

(51) Int. Cl. G16B 30/00; G16B 40/00

(52) U.S. Cl. CPC G16B 30/00; G16B 40/00

(57) ABSTRACT

The technology disclosed is directed to context-dependent base calling. The technology disclosed describes a system including memory storing k-mer-specific centroids for k-mers. The k-mer-specific centroids are learned by training a base calling pipeline to represent base calls of an already base called sequence in k-mer-specific time series, transform the k-mer-specific time series into predicted k-mer-specific centroids, merge the predicted k-mer-specific centroids on a sequencing cycle-by-sequencing cycle basis to generate predicted per-sequencing cycle intensity values, determine a training loss (e.g., a transformation loss) based on comparing the predicted per-sequencing cycle intensity values against known intensity values of the base calls, update the predicted k-mer-specific centroids based on the determined training loss, and store the updated centroids as the k-mer-specific centroids. The system also includes runtime logic that uses the k-mer-specific centroids to base call bases in a yet-to-be base called sequence in dependence upon k-mer context.

### CROSS-REFERENCE TO RELATED APPLICATIONS

The present application claims the benefit of, and priority to, U.S. Provisional Application 63/476,428, entitled “CONTEXT-DEPENDENT BASE CALLING,” filed Dec. 21, 2022. The aforementioned application is hereby incorporated by reference in its entirety.

### FIELD OF THE TECHNOLOGY DISCLOSED

The technology disclosed relates to apparatus and corresponding methods for the automated analysis of an image or recognition of a pattern. Included herein are systems that transform an image for the purpose of (a) enhancing its visual quality prior to recognition, (b) locating and registering the image relative to a sensor or stored prototype, or reducing the amount of image data by discarding irrelevant data, and (c) measuring significant characteristics of the image. In particular, the technology disclosed relates to segmenting clusters into subpopulations and base calling clusters in a particular subpopulation.

### Incorporations

The following are incorporated by reference for all purposes as if fully set forth herein:
U.S. Nonprovisional patent application Ser. No. 17/308,035, titled “EQUALIZATION-BASED IMAGE PROCESSING AND SPATIAL CROSSTALK ATTENUATOR,” filed May 4, 2021 (Attorney Docket No. ILLM 1032-2/IP-1991-US);
U.S. Provisional Patent Application No. 63/106,256, titled “SYSTEMS AND METHODS FOR PER-CLUSTER INTENSITY CORRECTION AND BASE CALLING,” filed on Oct. 27, 2020;
U.S. Nonprovisional patent application Ser. No. 15/909,437, titled “OPTICAL DISTORTION CORRECTION FOR IMAGED SAMPLES,” filed on Mar. 1, 2018;
U.S. Nonprovisional patent application Ser. No. 14/530,299, titled “IMAGE ANALYSIS USEFUL FOR PATTERNED OBJECTS,” filed on Oct. 31, 2014;
U.S. Nonprovisional patent application Ser. No. 15/153,953, titled “METHODS AND SYSTEMS FOR ANALYZING IMAGE DATA,” filed on Dec. 3, 2014;
U.S. Nonprovisional patent application Ser. No. 15/863,241, titled “PHASING CORRECTION,” filed on Jan. 5, 2018;
U.S. Nonprovisional patent application Ser. No. 14/020,570, titled “CENTROID MARKERS FOR IMAGE ANALYSIS OF HIGH DENSITY CLUSTERS IN COMPLEX POLYNUCLEOTIDE SEQUENCING,” filed on Sep. 6, 2013;
U.S. Nonprovisional patent application Ser. No. 12/565,341, titled “METHOD AND SYSTEM FOR DETERMINING THE ACCURACY OF DNA BASE IDENTIFICATIONS,” filed on Sep. 23, 2009;
U.S. Nonprovisional patent application Ser. No. 12/295,337, titled “SYSTEMS AND DEVICES FOR SEQUENCE BY SYNTHESIS ANALYSIS,” filed on Mar. 30, 2007;
U.S. Nonprovisional patent application Ser. No. 12/020,739, titled “IMAGE DATA EFFICIENT GENETIC SEQUENCING METHOD AND SYSTEM,” filed on Jan. 28, 2008;
U.S. Nonprovisional patent application Ser. No. 13/833,619, titled “BIOSENSORS FOR BIOLOGICAL OR CHEMICAL ANALYSIS AND SYSTEMS AND METHODS FOR SAME,” filed on Mar. 15, 2013, (Attorney Docket No. IP-0626-US);
U.S. Nonprovisional patent application Ser. No. 15/175,489, titled “BIOSENSORS FOR BIOLOGICAL OR CHEMICAL ANALYSIS AND METHODS OF MANUFACTURING THE SAME,” filed on Jun. 7, 2016, (Attorney Docket No. IP-0689-US);
U.S. Nonprovisional patent application Ser. No. 13/882,088, titled “MICRODEVICES AND BIOSENSOR CARTRIDGES FOR BIOLOGICAL OR CHEMICAL ANALYSIS AND SYSTEMS AND METHODS FOR THE SAME,” filed on Apr. 26, 2013, (Attorney Docket No. IP-0462-US);
U.S. Nonprovisional patent application Ser. No. 13/624,200, titled “METHODS AND COMPOSITIONS FOR NUCLEIC ACID SEQUENCING,” filed on Sep. 21, 2012, (Attorney Docket No. IP-0538-US);
U.S. Nonprovisional patent application Ser. No. 13/006,206, titled “DATA PROCESSING SYSTEM AND METHODS,” filed on Jan. 13, 2011;
U.S. Nonprovisional patent application Ser. No. 15/936,365, titled “DETECTION APPARATUS HAVING A MICROFLUOROMETER, A FLUIDIC SYSTEM, AND A FLOW CELL LATCH CLAMP MODULE,” filed on Mar. 26, 2018;
U.S. Nonprovisional patent application Ser. No. 16/567,224, titled “FLOW CELLS AND METHODS RELATED TO SAME,” filed on Sep. 11, 2019;
U.S. Nonprovisional patent application Ser. No. 16/439,635, titled “DEVICE FOR LUMINESCENT IMAGING,” filed on Jun. 12, 2019;
U.S. Nonprovisional patent application Ser. No. 15/594,413, titled “INTEGRATED OPTOELECTRONIC READ HEAD AND FLUIDIC CARTRIDGE USEFUL FOR NUCLEIC ACID SEQUENCING,” filed on May 12, 2017;
U.S. Nonprovisional patent application Ser. No. 16/351,193, titled “ILLUMINATION FOR FLUORESCENCE IMAGING USING OBJECTIVE LENS,” filed on Mar. 12, 2019;
U.S. Nonprovisional patent application Ser. No. 12/638,770, titled “DYNAMIC AUTOFOCUS METHOD AND SYSTEM FOR ASSAY IMAGER,” filed on Dec. 15, 2009;
U.S. Nonprovisional patent application Ser. No. 13/783,043, titled “KINETIC EXCLUSION AMPLIFICATION OF NUCLEIC ACID LIBRARIES,” filed on Mar. 1, 2013; and
U.S. Nonprovisional patent application Ser. No. 16/826,168, titled “ARTIFICIAL INTELLIGENCE-BASED SEQUENCING,” filed 21 Mar. 2020 (Attorney Docket No. ILLM 1008-20/IP-1752-PRV).

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Various protocols in biological or chemical research involve performing a large number of controlled reactions on local support surfaces or within predefined reaction chambers. The desired reactions may then be observed or detected, and subsequent analysis may help identify or reveal properties of chemicals involved in the reaction. For example, in some multiplex assays, an unknown analyte having an identifiable label (e.g., fluorescent label) may be exposed to thousands of known probes under controlled conditions. Each known probe may be deposited into a corresponding well of a microplate. Observing any chemical reactions that occur between the known probes and the unknown analyte within the wells may help identify or reveal properties of the analyte. Other examples of such protocols include known DNA sequencing processes, such as sequencing-by-synthesis or cyclic-array sequencing. In cyclic-array sequencing, a dense array of DNA features (e.g., template nucleic acids) are sequenced through iterative cycles of enzymatic manipulation. After each cycle, an image may be captured and subsequently analyzed with other images to determine a sequence of the DNA features.

As a more specific example, one known DNA sequencing system uses a pyrosequencing process and includes a chip having a fused fiber-optic faceplate with millions of wells. A single capture bead having clonally amplified sstDNA from a genome of interest is deposited into each well. After the capture beads are deposited into the wells, nucleotides are sequentially added to the wells by flowing a solution containing a specific nucleotide along the faceplate. The environment within the wells is such that if a nucleotide flowing through a particular well complements the DNA strand on the corresponding capture bead, the nucleotide is added to the DNA strand. A colony of DNA strands is called a cluster, and a cluster can include many (thousands of) nucleotides. Incorporation of the nucleotide into the cluster initiates a process that ultimately generates a fluorescent light signal. The system includes a CCD camera that is positioned directly adjacent to the faceplate and is configured to detect the light signals from the DNA clusters in the wells. Subsequent analysis of the images taken throughout the pyrosequencing process can determine a sequence of the genome of interest. Based on different fluorescent light signals of nucleotides adenine (A), cytosine (C), guanine (G), and thymine (T), the particular nucleotide incorporated into the DNA strand of the cluster can be identified. This identification process is also known as “base calling.”

One challenge with the analysis of image data is variation in intensity profiles of clusters being base called. This may cause a drop in data throughput and an increase in error rates of base calling during a sequencing run. There are many potential reasons for intensity profile variation. It may result from the chemistry modulation effects where the intensity profiles of clusters at a current sequencing cycle can be shifted based on their base context. It may result from differences in cluster brightness, caused by fragment length distribution in the cluster population. It may result from phasing, which occurs when a molecule in a cluster does not incorporate a nucleotide in some sequencing cycles and lags behind other molecules, or when a molecule incorporates more than one nucleotide in a single sequencing cycle. It may result from fading, i.e., exponential decay in signal intensity of clusters as a function of sequencing cycle number due to excessive washing and laser exposure as the sequencing run progresses. It may result from underdeveloped cluster colonies, i.e., small cluster sizes that produce empty or partially filled wells on a patterned flow cell. It may result from overlapping cluster colonies caused by unexclusive amplification. It may result from under illumination or uneven illumination, for example, due to clusters being located on the edges of a flow cell. It may result from impurities on a flow cell that obfuscate emitted signal. It may result from polyclonal clusters, i.e., when multiple clusters are deposited in the same well.

Base calling accuracy is crucial for high-throughput DNA sequencing and downstream analysis such as read mapping and genome assembly. Accordingly, an opportunity arises to correct the intensity variations of clusters. Improved base calling throughput and reduced base calling error rate during a sequencing run may result.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The color drawings also may be available in PAIR via the Supplemental Content tab. In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings, in which,

FIG. 1 illustrates a cross-section of an example biosensor that can be used in various embodiments;

FIG. 2 illustrates an example flow cell with eight lanes, and a zoom-in on one tile, in accordance with one or more embodiments of the technology disclosed;

FIG. 3 illustrates an example flow cell with eight lanes, and a zoom-in on one tile and its clusters and their surrounding background, in accordance with one or more embodiments of the technology disclosed;

FIG. 4 illustrates variations in the intensity profiles of clusters caused by different base context, in accordance with one or more embodiments of the technology disclosed;

FIG. 5 illustrates examples of k-mer-specific intensity distributions, in accordance with one or more embodiments of the technology disclosed;

FIG. 6 illustrates an example Context-Dependent Signal Modulation (CDSM) model 600 that takes base calls of known sequences as input and generates k-mer-specific centroids, in accordance with one or more embodiments of the technology disclosed;

FIG. 7 illustrates examples of encoded base calls at two color/intensity channels, in accordance with one or more embodiments of the technology disclosed;

FIG. 8 illustrates another example of encoded base calls at twenty sequencing cycles in a sequencing run at two color/intensity channels, in accordance with one or more embodiments of the technology disclosed;

FIGS. 9A-9D illustrate examples of k-mer-specific time series and transformations thereof, in accordance with one or more embodiments of the technology disclosed;

FIGS. 10A-10B illustrate examples of k-mer-specific time series before the transformation, in accordance with one or more embodiments of the technology disclosed;

FIGS. 11A-11B illustrates examples of k-mer-specific time series after the transformation, in accordance with one or more embodiments of the technology disclosed;

FIG. 12 illustrates a block diagram of training the CDSM model, in accordance with one or more embodiments of the technology disclosed;

FIG. 13A illustrates an example of generating predicted k-mer-specific centroids via the CDSM model and using the k-mer-specific centroids for base calling, in accordance with one or more embodiments of the technology disclosed;

FIG. 13B illustrates another example of generating predicted k-mer-specific centroids via the CDSM model and using the k-mer-specific centroids for base calling, in accordance with one or more embodiments of the technology disclosed;

FIG. 14 illustrates comparisons between predicted intensities by the base calling pipeline and observed intensities extracted from sequencing images captured from the first color/intensity channel at each sequencing cycle, in accordance with one or more embodiments of the technology disclosed;

FIG. 15 illustrates comparisons in signal-to-noise (SNRs) ratios of context-independent base calling and context-dependent base calling over a plurality of sequencing cycles of a sequencing run, in accordance with one or more embodiments of the technology disclosed;

FIGS. 16A-16D illustrate comparisons between the intensity distributions of a single cluster without and with corrections for context-dependent effects over a plurality of sequencing cycles of a sequencing run, where the first row is model output, the second row is model input, the left column is a simulated sequence through the model showing that the characteristic spread of the clouds has been properly captured in the model, and the right column shows an actual signal from the sequencer (top) and its sequence dependent modulated correction (bottom) in accordance with one or more embodiments of the technology disclosed;

FIGS. 17A-17B illustrate comparisons between the intensity distribution of a plurality of clusters without and with correction for context-dependent effects, in accordance with one or more embodiments of the technology disclosed;

FIGS. 18A-18D illustrate examples of tetramer-specific matrices that transform tetramer-specific time series to predicted tetramer-specific centroids, in accordance with one or more embodiments of the technology disclosed;

FIGS. 19A-19C depict correspondence between identified tetramers and corresponding error rates improvements uncovered in a different deep learning basecaller when base calling clusters with the identified tetramer context, in accordance with one or more embodiments of the technology disclosed;

FIGS. 20A-20B illustrate an example of phasing and prephasing effects;

FIG. 21A illustrates an example of fading, in which signal intensity is decreased as a function of cycle number is a sequencing run of a base calling operation;

FIG. 21B conceptually illustrates a decreasing signal-to-noise ratio as cycles of sequencing progress; and

FIG. 22 illustrates a computer system that can be used to implement the technology disclosed.

### DETAILED DESCRIPTION

The following discussion is presented to enable any person skilled in the art to make and use the technology disclosed and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown but is to be accorded the widest scope consistent with the principles and features disclosed herein.

The discussion is organized as follows. First, we introduce base calling clusters and variations in intensity profiles of the clusters caused by base context. Then we propose the technology disclosed for a base calling pipeline that processes base calls of an already based called sequence and is iteratively trained to generate predicted k-mer-specific centroids. Each of the k-mer-specific centroids represents a mean value of the intensities of clusters with the same k-mer context. In particular, the base calling pipeline includes a context-dependent signal modulation model that subdivides base calls of already base called sequences into k-mer-specific time series, transforms these time series and merges them into predicted per-sequencing cycle intensity values represented by k-mer-specific centroids. After that, we setup examples of using k-mer-specific centroids to base call target clusters. Advancing further, we provide various performance results of context-dependent base calling and improvement over context-independent base calling approaches.

### Introduction

The technology disclosed begins with the concept of clusters, intensity extraction and base calling clusters. In one implementation, a sequencer uses sequencing by synthesis (SBS) technology for generating sequencing images. SBS relies on growing nascent strands complementary to cluster strands with fluorescently-labeled nucleotides, while tracking the emitted signal of each newly added nucleotide. The fluorescently-labeled nucleotides have a 3′ removable block that anchors a fluorophore signal of the nucleotide type. SBS occurs in repetitive sequencing cycles, each comprising three steps: (a) extension of a nascent strand by adding the fluorescently-labeled nucleotide; (b) excitation of the fluorophore using one or more lasers of an optical system of the sequencer and imaging through different filters of the optical system, yielding sequencing images; and (c) cleavage of the fluorophore and removal of the 3′ block in preparation for the next sequencing cycle. Incorporation and imaging are repeated up to a designated number of sequencing cycles, defining the read length, which refers to the number of base pairs (bp) sequenced from a DNA fragment. Using this approach, each sequencing cycle interrogates a new position along the cluster strands.

Intensity values can be extracted from different color/intensity channel sequencing images generated by a sequencer at each sequencing cycle during a sequencing run. Examples of the sequencer include Illumina's iSeq, HiSeqX, HiSeq 3000, HiSeq 4000, HiSeq 2500, NovaSeq 6000, NextSeq 550, NextSeq 1000, NextSeq 2000, NextSeqDx, MiSeq, and MiSeqDx.

The tremendous power of Illumina's sequencers stems from their ability to simultaneously execute and sense millions or even billions of analytes (e.g., clusters). A cluster comprises approximately one thousand identical copies of a template strand, though clusters vary in size and shape. Clusters are grown from the template strand, prior to the sequencing run, by bridge amplification or exclusion amplification of the input library which is a collection of similarly sized DNA fragments. The purpose of the amplification and cluster growth is to increase the intensity of the emitted signal since the imaging device cannot reliably sense the fluorophore signal of a single strand. In some embodiments, the imaging device perceives a cluster of thousands of template strands as a single spot. For instance, the imaging device can detect such a cluster of thousands of template strands as a spot represented by a single pixel or multiple pixels.

The sequencing process occurs in a flow cell—a small glass slide that holds the input DNA fragments during the sequencing process. The flow cell is connected to the high-throughput optical system that includes microscopic imaging, excitation lasers, and fluorescence filters. In some cases, the flow cell consists of (or includes) a complementary metal-oxide-semiconductor (CMOS). An imaging device (e.g., a solid-state imager such as a charge-coupled device (CCD) or a CMOS sensor) in the sequencer takes images at multiple locations along a series of non-overlapping regions called tiles. At each sequencing cycle, the imaging device takes sequencing images of each tile at each color/intensity channel. The sequence data of clusters immobilized on each tile at each sequencing cycle, therefore, includes intensity signals extracted from the sequencing images.

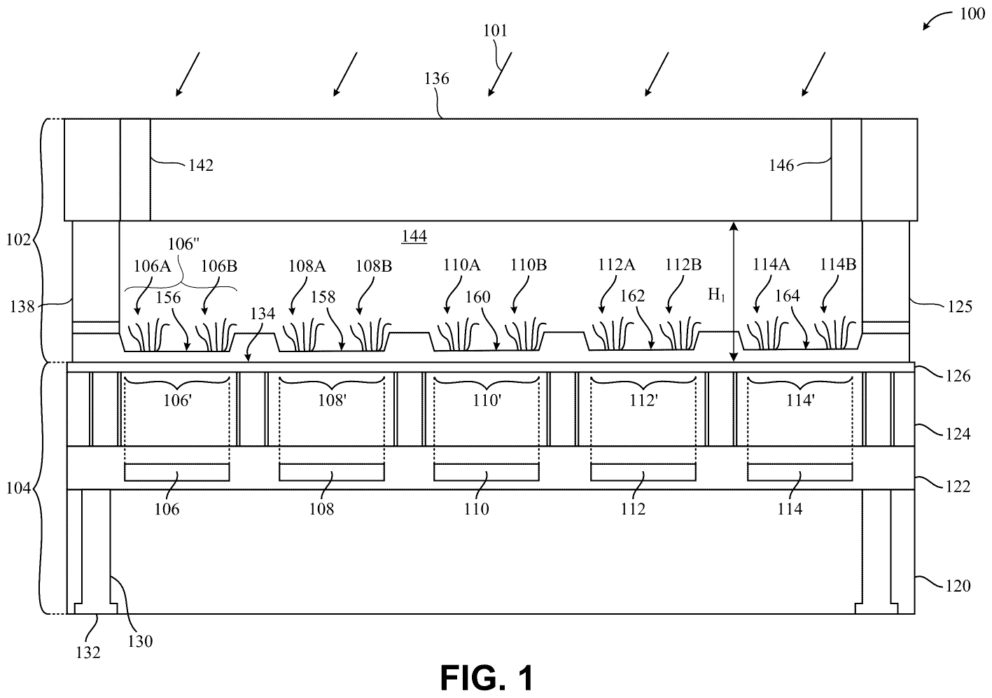

FIG. 1 illustrates a cross-section of a biosensor 100 that can be used in various embodiments. Biosensor 100 has pixel areas 106′, 108′, 110′, 112′, and 114′ that each can hold more than one cluster during a base calling cycle (e.g., 2 clusters per pixel area). As shown, the biosensor 100 includes a flow cell 102 that is mounted onto a sampling device 104. In the illustrated embodiment, the flow cell 102 is affixed directly to the sampling device 104. However, in alternative embodiments, the flow cell 102 may be removably coupled to the sampling device 104. The sampling device 104 has a sample surface 134 that may be functionalized (e.g., chemically or physically modified in a suitable manner for conducting the desired reactions). For example, the sample surface 134 may be functionalized and may include a plurality of pixel areas 106′, 108′, 110′, 112′, and 114′ that can each hold more than one cluster during a base calling cycle (e.g., each having a corresponding cluster pair 106A, 106B; 108A, 108B; 110A, 110B; 112A, 112B; and 114A, 114B immobilized thereto). Each pixel area is associated with a corresponding sensor (or pixel or photodiode) 106, 108, 110, 112, and 114, such that light received by the pixel area is captured by the corresponding sensor. A pixel area 106′ can be also associated with a corresponding reaction site 106″ on the sample surface 134 that holds a cluster pair, such that light emitted from the reaction site 106″ is received by the pixel area 106′ and captured by the corresponding sensor 106. As a result of this sensing structure, in the case in which two or more clusters are present in a pixel area of a particular sensor during a base calling cycle (e.g., each having a corresponding cluster pair), the pixel signal in that base calling cycle carries information based on all of the two or more clusters. As a result, signal processing as described herein is used to distinguish each cluster, where there are more clusters than pixel signals in a given sampling event of a particular base calling cycle.

In the illustrated embodiment, the flow cell 102 includes sidewalls 138, 125, and a flow cover 136 that is supported by the sidewalls 138, 125. The sidewalls 138, 125 are coupled to the sample surface 134 and extend between the flow cover 136 and the sidewalls 138, 125. In some embodiments, the sidewalls 138, 125 are formed from a curable adhesive layer that bonds the flow cover 136 to the sampling device 104.

The sidewalls 138, 125 are sized and shaped so that a flow channel 144 exists between the flow cover 136 and the sampling device 104. The flow cover 136 may include a material that is transparent to excitation light 101 propagating from an exterior of the biosensor 100 into the flow channel 144. In an example, the excitation light 101 approaches the flow cover 136 at a non-orthogonal (or orthogonal) angle.

Also shown, the flow cover 136 may include inlet and outlet ports 142, 146 that are configured to fluidically engage other ports (not shown). For example, the other ports may be from the cartridge or the workstation. The flow channel 144 is sized and shaped to direct a fluid along the sample surface 134. A height H₁ and other dimensions of the flow channel 144 may be configured to maintain a substantially even flow of a fluid along the sample surface 134. The dimensions of the flow channel 144 may also be configured to control bubble formation.

By way of example, the flow cover 136 (or the flow cell 102) may comprise a transparent material, such as glass or plastic. The flow cover 136 may constitute a substantially rectangular block having a planar exterior surface and a planar inner surface that defines the flow channel 144. The block may be mounted onto the sidewalls 138, 125. Alternatively, the flow cell 102 may be etched to define the flow cover 136 and the sidewalls 138, 125. For example, a recess may be etched into the transparent material. When the etched material is mounted to the sampling device 104, the recess may become the flow channel 144.

The sampling device 104 may be similar to, for example, an integrated circuit comprising a plurality of stacked substrate layers 120-126. The substrate layers 120-126 may include a base substrate 120, a solid-state imager 122 (e.g., CMOS image sensor), a filter or light-management layer 124, and a passivation layer 126. It should be noted that the above is only illustrative and that other embodiments may include fewer or additional layers. Moreover, each of the substrate layers 120-126 may include a plurality of sub-layers. The sampling device 104 may be manufactured using processes that are similar to those used in manufacturing integrated circuits, such as CMOS image sensors and CCDs. For example, the substrate layers 120-126 or portions thereof may be grown, deposited, etched, and the like to form the sampling device 104.

The passivation layer 126 is configured to shield the filter layer 124 from the fluidic environment of the flow channel 144. In some cases, the passivation layer 126 is also configured to provide a solid surface (i.e., the sample surface 134) that permits biomolecules or other analytes-of-interest to be immobilized thereon. For example, each of the reaction sites may include a cluster of biomolecules that are immobilized to the sample surface 134. Thus, the passivation layer 126 may be formed from a material that permits the reaction sites to be immobilized thereto. The passivation layer 126 may also comprise a material that is at least transparent to a desired fluorescent light. By way of example, the passivation layer 126 may include silicon nitride (Si₂N₄) and/or silica (SiO₂). However, other suitable material(s) may be used. In the illustrated embodiment, the passivation layer 126 may be substantially planar. However, in alternative embodiments, the passivation layer 126 may include recesses, such as pits, wells, grooves, and the like. In the illustrated embodiment, the passivation layer 126 has a thickness that is about 150-200 nm and, more particularly, about 170 nm.

The filter layer 124 may include various features that affect the transmission of light. In some embodiments, the filter layer 124 can perform multiple functions. For instance, the filter layer 124 may be configured to (a) filter unwanted light signals, such as light signals from an excitation light source; (b) direct emission signals from the reaction sites toward corresponding sensors 106, 108, 110, 112, and 114 that are configured to detect the emission signals from the reaction sites; or (c) block or prevent detection of unwanted emission signals from adjacent reaction sites. As such, the filter layer 124 may also be referred to as a light-management layer. In the illustrated embodiment, the filter layer 124 has a thickness that is about 1-5 μm and, more particularly, about 2-4 μm. In alternative embodiments, the filter layer 124 may include an array of microlenses or other optical components. Each of the microlenses may be configured to direct emission signals from an associated reaction site to a sensor.

In some embodiments, the solid-state imager 122 and the base substrate 120 may be provided together as a previously constructed solid-state imaging device (e.g., CMOS chip). For example, the base substrate 120 may be a wafer of silicon and the solid-state imager 122 may be mounted thereon. The solid-state imager 122 includes a layer of semiconductor material (e.g., silicon) and the sensors 106, 108, 110, 112, and 114. In the illustrated embodiment, the sensors are photodiodes configured to detect light. In other embodiments, the sensors comprise light detectors. The solid-state imager 122 may be manufactured as a single chip through CMOS-based fabrication processes.

The solid-state imager 122 may include a dense array of sensors 106, 108, 110, 112, and 114 that are configured to detect activity indicative of a desired reaction from within or along the flow channel 144. In some embodiments, each sensor has a pixel area (or detection area) that is about 1-2 square micrometer (μm²). The array can include 500,000 sensors, 5 million sensors, 10 million sensors, or even 200 million sensors. The sensors 106, 108, 110, 112, and 114 can be configured to detect a predetermined wavelength of light that is indicative of the desired reactions.

In some embodiments, the sampling device 104 includes a microcircuit arrangement, such as the microcircuit arrangement described in U.S. Pat. No. 7,595,882, which is incorporated herein by reference in the entirety. More specifically, the sampling device 104 may comprise an integrated circuit having a planar array of the sensors 106, 108, 110, 112, and 114. Circuitry formed within the sampling device 104 may be configured for at least one of signal amplification, digitization, storage, and processing. The circuitry may collect and analyze the detected fluorescent light and generate pixel signals (or detection signals) for communicating detection data to a signal processor. The circuitry may also perform additional analog and/or digital signal processing in the sampling device 104. Sampling device 104 may include conductive vias 130 that perform signal routing (e.g., transmit the pixel signals to the signal processor). The pixel signals may also be transmitted through electrical contacts 132 of the sampling device 104.

The sampling device 104 is discussed in further detail with respect to U.S. Nonprovisional patent application Ser. No. 16/874,599, titled “Systems and Devices for Characterization and Performance Analysis of Pixel-Based Sequencing,” filed May 14, 2020, which is incorporated by reference as if fully set forth herein. The sampling device 104 is not limited to the above constructions or uses as described above. In alternative embodiments, the sampling device 104 may take other forms. For example, the sampling device 104 may comprise a CCD device, such as a CCD camera, that is coupled to a flow cell or is moved to interface with a flow cell having reaction sites therein.

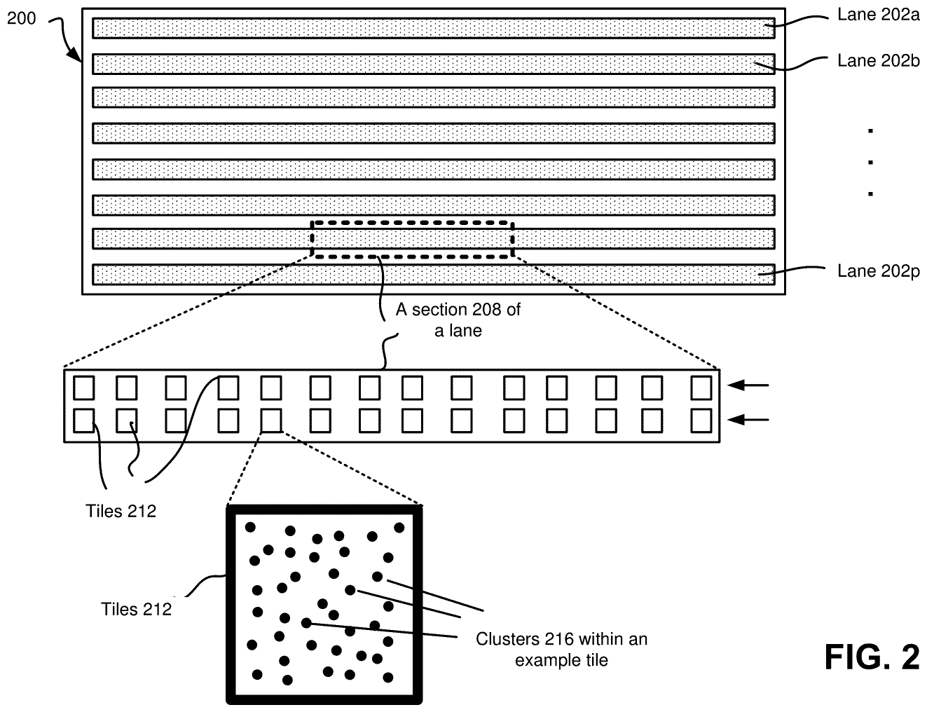

FIG. 2 depicts an example flow cell 200 where clusters 216 are immobilized and base called during a sequencing process. In one implementation, the flow cell 200 is partitioned in a plurality of chambers called lanes, such as lanes 202a, 202b, . . . , 202p, i.e., p represents a number of lanes. The lanes are physically separated from each other and may contain different tagged sequencing input libraries, distinguishable without sample cross-contamination. Each individual lane 202 can further be partitioned into non-overlapping regions called “tiles” 212. For example, FIG. 2 illustrates a magnified view of section 208 of an example lane. Section 208 is illustrated to comprise a plurality of tiles 212. Hundreds of thousands to millions of clusters 216 can be immobilized on the surface of each tile. At each sequencing cycle of a sequencing run, the imaging device of the sequencer takes sequencing images of each tile at each color/intensity channel. The intensity profiles of clusters being base called at each sequencing cycle are extracted from the sequencing images and analyzed for base calling.

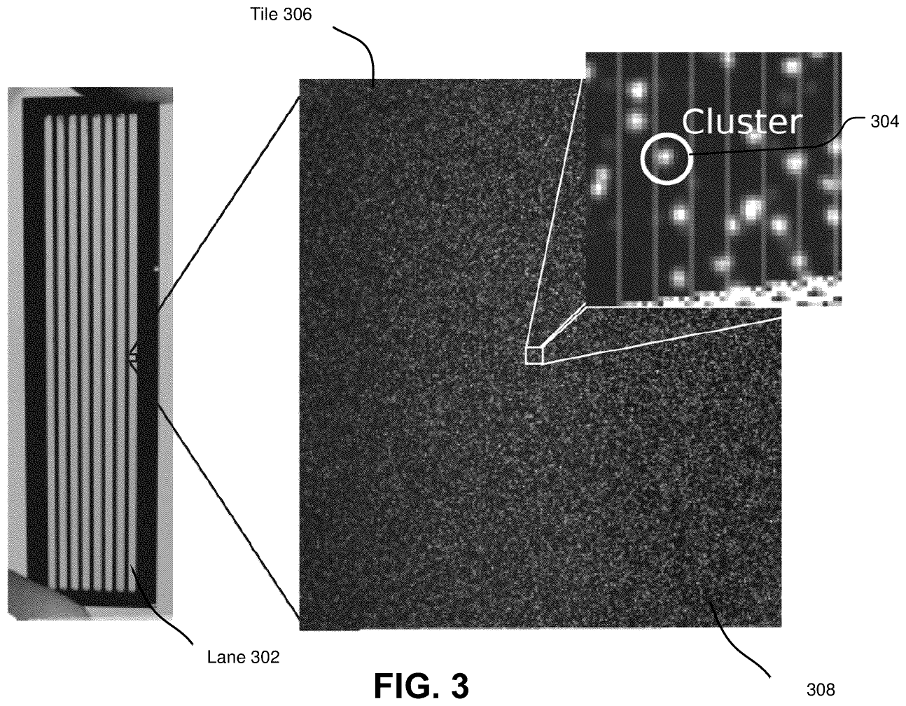

FIG. 3 illustrates an example Illumina GA-Iix™ flow cell with eight lanes 302, and also illustrates a zoom-in on one tile 306 and its clusters and their surrounding background. For example, there are a hundred tiles per lane in Illumina Genome Analyzer II and sixty-eight tiles per lane in Illumina HiSeq2000. A tile 306 holds hundreds of thousands to millions of clusters. In FIG. 3, an image generated from the tile 306 with clusters shown as bright spots is shown at 308 (e.g., 308 is a magnified image view of a tile), with an example cluster 304 labeled. A cluster 304 comprises approximately one thousand identical copies of a template molecule, though clusters vary in size and shape. The clusters are grown from the template molecule, prior to the sequencing run, by bridge amplification of the input library. The purpose of the amplification and cluster growth is to increase the intensity of the emitted signal since the imaging device cannot reliably sense a single fluorophore. However, the physical distance of the DNA fragments within a cluster 304 is small, so the imaging device perceives the cluster of fragments as a single spot 304.

The clusters and the tiles are discussed in further detail with respect to U.S. Nonprovisional patent application Ser. No. 16/825,987, titled “Training Data Generation For Artificial Intelligence-based Sequencing,” filed 20 Mar. 2020.

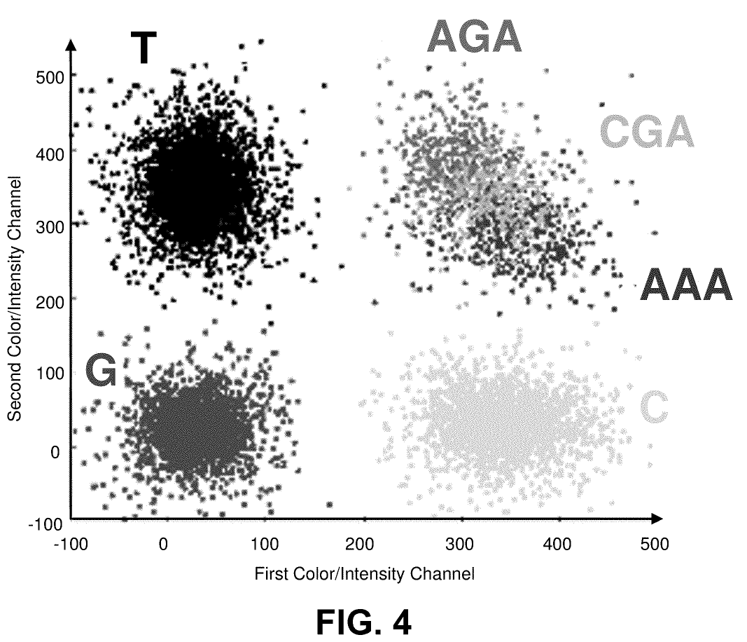

FIG. 4 illustrates variations in the intensity profiles of clusters caused by different base context. The intensity profiles of clusters represent intensity values that capture the fluorescent signals produced due to nucleotide incorporations in the clusters at a plurality of sequencing cycles during a sequencing run. Each data point in FIG. 4 represents the intensity profiles of a cluster at a given sequencing cycle. The identity of four different nucleotide types/bases adenine (A), cytosine (C), guanine (G), and thymine (T) is encoded as a combination of the intensity values in two-color images, i.e., the first and second color/intensity channels. For example, a nucleic acid can be sequenced by providing a first nucleotide type (e.g., base C) that is detected at the first color/intensity channel, a second nucleotide type (e.g., base T) that is detected at the second color/intensity channel, a third nucleotide type (e.g., base A) that is detected at both the first and the second color/intensity channels, and a fourth nucleotide type (e.g., base G) that lacks a label that is not, or minimally, detected at either color/intensity channel. The intensity values captured at the first color/intensity channel are plotted against the intensity values at the second color/intensity channel (e.g., as a scatterplot), and therefore, the intensity values are segregated into four intensity distributions.

Base calling can be performed by fitting a mathematical model to the intensity profiles of clusters to be called. In particular, a mixture of four intensity distributions can be fitted to the intensity values of a target cluster to be called at a given sequencing cycle and determines the likelihoods of the intensity profiles of the target cluster belonging to each of the four intensity distributions.

In some implementations, the mixture of intensity distribution is a Gaussian mixture model. A Gaussian mixture model comprises multiple Gaussians, each identified by k ∈{1, . . . , K}, where K is the number of clusters (i.e., groups of data points). For example, the Gaussian mixture model can include four intensity distributions, corresponding to four nucleotide bases A, G, C and T. Each Gaussian k in the mixture includes the following parameters:
A mean value μ that defines its centroid; and
Covariances Σ that define its width. In a multivariate scenario where, e.g., the intensity profiles for the clusters are extracted from the sequencing images acquired from two color/intensity channels, the covariances Σ define the dimension of an ellipsoid of the intensity distribution.

When a target cluster is to be base called at a current sequencing cycle, an expectation maximization algorithm can be used to fit the mixture of intensity distributions to the intensity profiles of the target cluster during the current sequencing cycle. When the mixture of intensity distributions is a Gaussian mixture model, for example, the expectation maximization algorithm iteratively maximizes the likelihood of observing means u (centroids) and covariances Σ (dimensions of the ellipsoid) that best fit the intensity profiles for the target cluster to be base called. For each of the four intensity distributions corresponding to one of the four bases A, C, T, and G, a centroid and covariances of the distribution are calculated. The intensity distribution (or centroid of the intensity distribution) with a maximum likelihood to which the target cluster belongs is determined as the base call for the target cluster.

For a base to be called at a given sequencing cycle, its corresponding base context varies. Base context refers to prior and/or succeeding bases that are identified at prior and/or succeeding sequencing cycles, respectively. Analysis has revealed that the intensity profiles of clusters at a current sequencing cycle can be shifted based on their base context identified at prior and succeeding sequencing cycles, also known as chemistry modulation effects or fully functional nucleotide (FFN) triphosphate modulation effects. In some embodiments, chemistry modulation effects result from differential incorporation of two (or more) FFN species for a given base. When prior base context includes one or more base A, the shift in the intensity distribution can be substantial. As illustrated in FIG. 4, the clusters that are called as base A at a given sequencing cycle have different base context, namely, AGA, CGA and AAA. The prior bases AG, CG and AA are identified at prior sequencing cycles. The chemistry modulation effects caused by different prior bases AG, CG and AA lead to substantial variation in the intensity profiles at both color/intensity channels. These base context-specific variations can cause miscalls, especially when the intensity profile of a target cluster to be called is close to a decision boundary, i.e., between two intensity distributions of different bases, for example, bases A and C, bases A and T.

Quenching effect is another effect by which base context causes variations in the intensity profiles of clusters. In the sequencing-by-synthesis (SBS) process, nucleotides incorporated into the template sequences contain fluorophores that specifically identify the types of the bases, and attached to the nucleotides is a cleavable linker. After the incorporated base is identified, the linker is cleaved, allowing the fluorophore to be removed and ready for the next base to be attached and identified. Nevertheless, the cleavage can leave a remaining “pendant arm” moiety located on each of the detected nucleotides, which impacts the intensity profiles of the following nucleotides incorporated into the template sequences. For example, the remaining “pendant arm” after the cleavage of the fluorophores attached to base G quenches/reduces/suppresses the intensity values of a subsequent fluorophore when the next nucleotide is incorporated. The quenching effect can be substantial when base calling dimer GA. The fluorophores attached to base A can be significantly quenched by the “pendant arm” of the fluorophores attached to prior base G. In a two-channel base calling system, the intensity values of base A at both color/intensity channels can be reduced, increasing the risk of miscalls. The intensity profiles of other bases (e.g., C, G and T) can be similarly impacted by the “pendant arm” of the fluorophores attached to base G (or some other nucleotide base). In some cases, however, a preceding G can lead to a high average intensity in certain FFN sets, while an A directly preceding an A can lead to relatively low intensity values.

### Context-Dependent Base Calling

The technology disclosed provides approaches to context-dependent base calling, by taking into consideration the variations in the intensity profiles of clusters caused by their base context. We introduce a base calling system including memory storing context-specific centroids and runtime logic configured to use the context-specific centroids to base call a target cluster. Each of the context-specific centroids represents a mean value μ of the intensity distribution of clusters with the same base context.

Base context can be represented by k-mers (k≥1). The k-mers can be 4∧k permutations of k base positions, where 4 corresponds to four bases A, G, C and T. Therefore, context-specific centroid can be k-mer-specific centroids, including 4∧k k-mer-specific centroids. Each k-mer-specific centroid can represent the mean value μ of the intensity distributions of clusters with the same k-mer context.

The context-specific centroids can be learned by iteratively training a base calling pipeline using base calls of known (i.e., already base called) sequences as training samples. In one or more embodiments, the base calling pipeline can process the base calls of already base called sequences in k-mer-specific time series, each of the k-mer-specific time series representing presence or absence of a particular k-mer at each sequencing cycle in a plurality of sequencing cycles across which the base calls are generated. The base calling pipeline can transform the k-mer-specific time series into predicted k-mer-specific centroids and merge these predicted k-mer-specific centroids on a sequencing cycle-by-sequencing cycle basis to generate predicted per-sequencing cycle intensity values represented by the predicted k-mer-specific centroids. By comparing the predicted per-sequencing cycle intensity values against known intensity values of the base calls, the base calling pipeline can determine a training loss (e.g., a transformation loss) and based on which, update the predicted k-mer-specific centroids accordingly to generate updated k-mer-specific centroids. These updated k-mer-specific centroids can be stored in the memory as k-mer-specific centroids.

The base calling system can use context-specific centroids to base call a target cluster. The base calling system can access current intensity data of the target cluster captured at a current sequencing cycle of a sequencing run, as well as context intensity data of the target cluster for at least one of a preceding sequencing cycle or a succeeding sequencing cycle. The context intensity data is used to identify the base context of the target cluster. For instance, the base calling system can determine base context from prior and/or succeeding base calls based on base calls made during previous cycles (e.g., prior base calls) and/or preliminary base calls made for future cycles (e.g., succeeding base calls). The base calling system can access k-mer-specific centroids stored in the memory and select context-specific centroids that correspond to the base context of the target cluster. By comparing the current intensity data of the target cluster with the selected context-specific centroids, the base calling system can base call the cluster. For example, the context-specific centroid of the intensity distribution with a maximum likelihood to which the target cluster belongs can be determined as the base call for the target cluster. Alternatively, one of the selected context-specific centroids that is closest to the current intensity data of the target cluster can be determined as the base call.

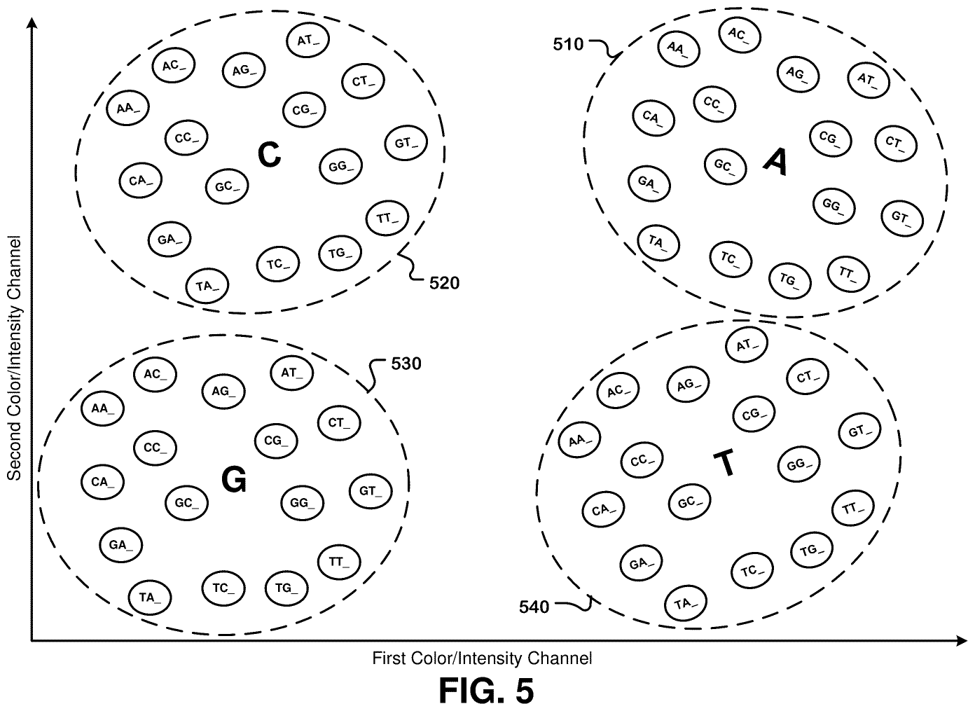

FIG. 5 illustrates examples of k-mer-specific intensity distributions. When k=3, for example, the mixture of intensity distributions includes sixty-four distributions, corresponding to sixty-four combinations of base context at three consecutive sequencing cycles N−2, N−1 and N. The sixty-four distributions can be categorized into four categories, each category corresponding to one of the four bases A, G, C and T at a current sequencing cycle N. Category A 510 corresponds to those clusters that are base called as A at the current sequencing cycle N. Category C 520 corresponds to those clusters that are based called as C at the current sequencing cycle N. Category G 530 corresponds to those clusters that are base called as G at the current sequencing cycle N. Category T 540 corresponds to those clusters that are based called as T at the current sequencing cycle N. Each of the four categories 510, 520, 530 and 540 includes sixteen distributions, corresponding two particular prior base calls (base context) identified at prior sequencing cycles N−2 and N−1. Category A 510, representing clusters that are base called as A at the current sequencing cycle N, includes sixteen distributions of combinations of two prior base calls AA_, AG_, AC_, AT_, CA_, CG_, CC_, CT_, GA_, GG_, GC_, GT_, TA, TG_, TC_ and TT_.

When a target cluster is to be called at the current sequencing cycle N, the base calling system can identify the corresponding base context determined from prior sequencing cycles. For example, the prior two bases that are called at prior sequencing cycles N−2 and N−1 can be G and A, respectively. The base calling system can select four intensity distributions, each with an optimized trimer-specific centroid, corresponding to the base context of GA_ (e.g., trimers GAA, GAG, GAC, and GAT). The base calling system can base call the target cluster at the current sequencing cycle by comparing the intensity profile of the cluster with the four centroids. In one or more embodiments, the base calling system calculates a Euclidean distance between each of the four trimer-specific centroids and the intensity profile of the target cluster at the respective color/intensity channel. The centroid of the intensity distribution with a shortest Euclidean distance to the target cluster is determined as the base call. Alternatively, the base calling system can determine the likelihoods of the intensity profiles of the target cluster belonging to each of the four intensity distributions. The centroid with a maximum likelihood to which the target cluster belongs is determined as the base call for the target cluster.

The k-mers can include a current base to be called at a current sequencing cycle and prior bases identified at prior sequencing cycles. When the base context is represented by dimers including a current base to be called and an immediately prior base (i.e., k=2), for example, there are sixteen (4∧2) permutations of two base positions, namely, AA, AG, AC, AT, CA, CG, CC, CT, GA, GG, GC, GT, TA, TG, TC and TT. Accordingly, there are sixteen (4∧2) dimer-specific centroids that can be learned by iteratively training the base calling pipeline. Consider a target cluster that is to be called at a given sequencing cycle with an immediately prior base A. To base call the target cluster, the base calling system can identify the immediately prior base A and select four dimer-specific centroids of the intensity distributions corresponding to dimer context AA, AG, AC and AT, respectively. The base calling system can compare the intensity profile of the target cluster at the current sequencing cycle with the four centroids and call the base for the target cluster.

As illustrated in FIG. 5, when the base context is represented by trimers including the current base to be called and two prior bases (i.e., k=3), there are sixty-four (4∧3) permutations of three base positions, AAA, ACA, AGA, ATA, CAA, CCA, CGA, CTA, GAA, GCA, GGA, GTA, TAA, TCA, TGA, TTA, AAC, ACC, AGC, ATC, CAC, CCC, CGC, CTC, GAC, GCC, GGC, GTC, TAC, TCC, TGC, TTC, AAG, ACG, AGG, ATG, CAG, CCG, CGG, CTG, GAG, GCG, GGG, GTG, TAG, TCG, TGG, TTG, AAT, ACT, AGT, ATT, CAT, CCT, CGT, CTT, GAT, GCT, GGT, GTT, TAT, TCT, TGT and TTT. Accordingly, there are sixty-four (4∧3) trimer-specific centroids that can be learned by iteratively training the base calling pipeline.

Alternatively, k-mers can include a current base to be called at a current sequencing cycle, prior bases identified at prior sequencing cycles and succeeding bases identified succeeding sequencing cycles. For example, base context can be represented by trimers including the current base to be called, one prior base and one succeeding base (i.e., k=3). Thus, there are sixty-four (4∧3) permutations of 3 base positions. Consider a target cluster that has been base called for three successive sequencing cycles, namely, cycles N−1, N, and N+1. The base calling system can determine a preliminary base call for the target cluster at each of the three successive sequencing cycles based on the corresponding intensity profiles. When the target cluster has a preliminary base A called at sequencing cycle N−1 and a preliminary base T called at sequencing cycle N+1, the base calling system can identify the base context A_T and compare the intensity profiles of the target cluster at the current sequencing cycle N with the trimer-specific centroids of the intensity distributions corresponding to base context AAT, AGT, ACT and ATT, respectively. In the foregoing trimer examples AAT, AGT, ACT and ATT, the base call in the middle of each trimer represents the base call for cycle N.

We now turn to the advantages of context-dependent base calling. In the base calling domain, sequencing characteristics can show significant diversity in various categories, including sequencing platforms, sequencing instruments, sequencing protocols, sequencing chemistries, sequencing reagents, cluster densities and so on. The disclosed base calling pipeline can be trained on large-scale training samples with diverse sequencing characteristics that adequately model the real-world sequencing runs.

More importantly, the disclosed base calling pipeline models context-dependent effects by iteratively learning context-specific centroids using large-scale known sequences as training samples. The optimized context-specific centroids accurately reflect the intensities of clusters having the same context but diverse sequencing characteristics. In other words, instead of generating a mixture of four intensity distribution with varying shapes and dimensions, the base calling pipeline can granulize them into groups of context-dependent distributions. It reduces the adverse impact of the intensity variations caused by e.g., chemistry modulation effects, FFN modulation effects, quenching effects and therefore, reduces the error rate of base calling.

The modeling of context-dependent effect can be trained offline to determine optimized context-specific centroids and thus, significantly saves computation power. Compared to base calling algorithms that rely on iteratively fitting both the centroids and covariances of the intensity distributions to the intensity profiles for a target cluster, the base call system disclosed herein may only need to compare four context-specific centroids with the intensity profiles of the target cluster for base calling at each sequencing cycle. Because each centroid is optimized to represent mean values of the intensity distributions of clusters with the same context, the corresponding intensity distribution can be considered substantially uniform (circular instead of elliptical). Therefore, the context-dependent base calling disclosed herein can improve the efficiency of base calling while maintaining the low error rate.

### Context-Dependent Signal Modulation (CDSM) Model

In one or more embodiments, the base calling pipeline is a context-dependent signal modulation (CDSM) model that corrects for the context-dependent effect. In some embodiments, the CDSM model functions by processing data from previously determined base calls for certain sequences. As described above, to “base call” a cluster at a given sequencing cycle refers to processing the intensity profiles of the cluster by fitting a mixture of intensity distributions to the intensity profiles and determines the base incorporated into the template nucleotide as one of the four bases A, G, C and T. After such initial base calling, the CDSM model takes as input base calls of known sequences and generates k-mer-specific centroids as predicted mean values of intensity distributions of clusters with k-mer-specific base context.

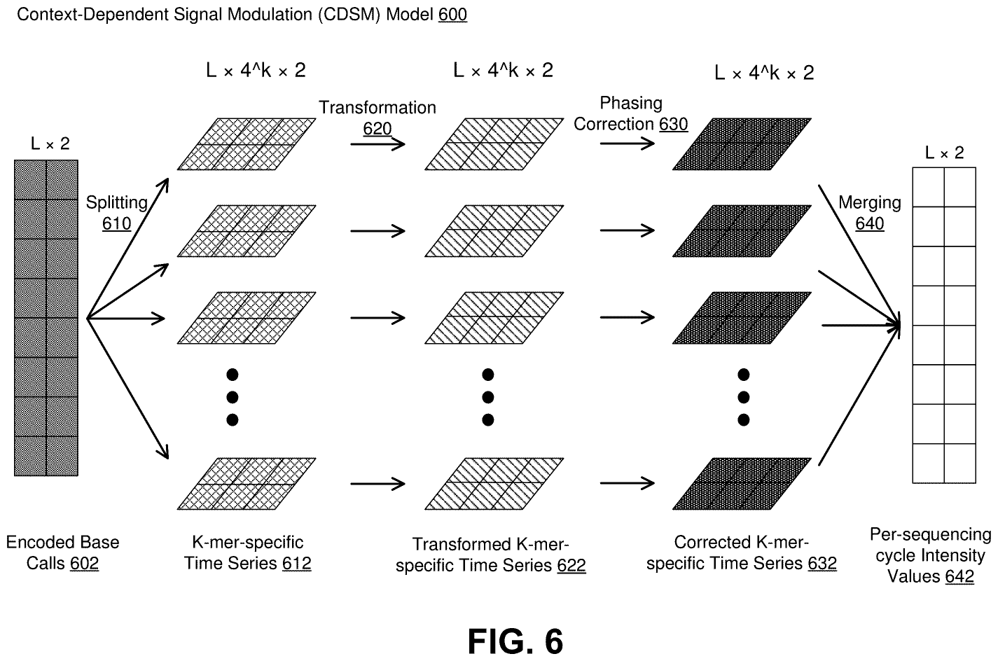

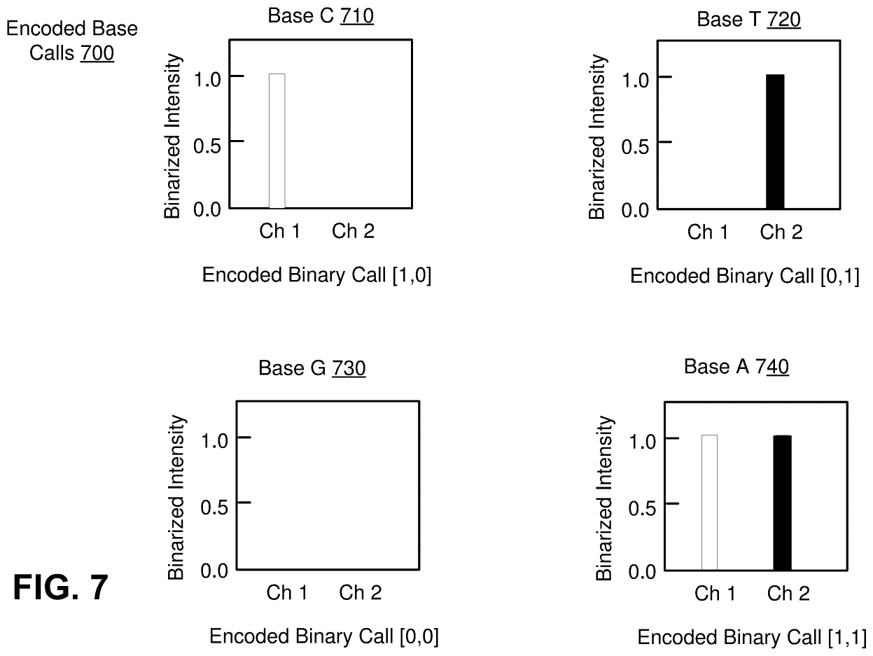

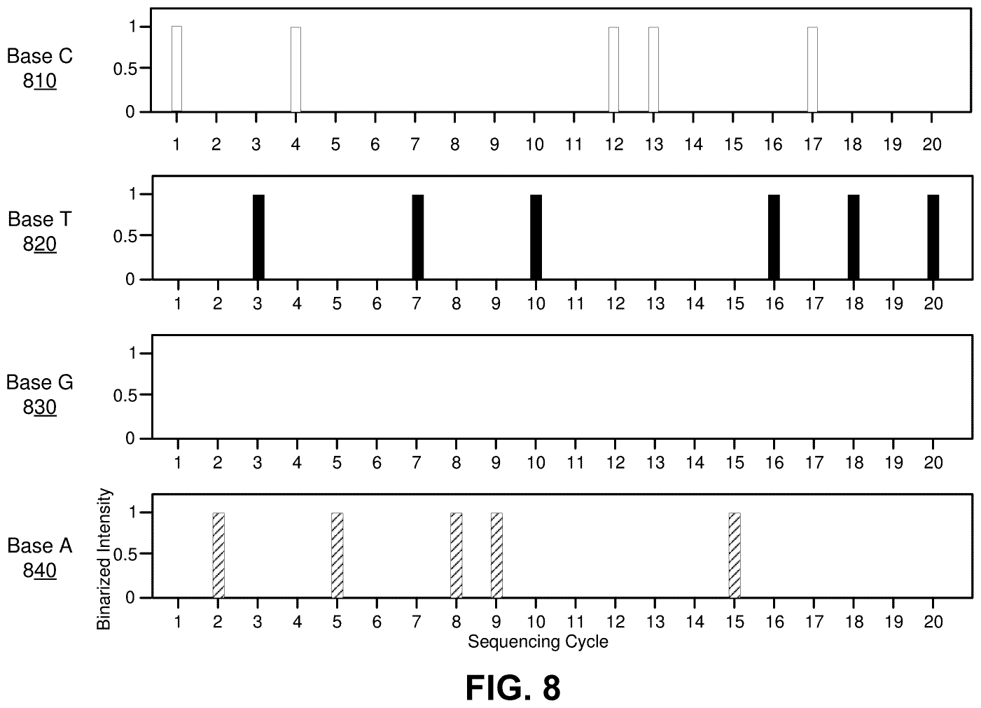

FIG. 6 illustrates an example CDSM model 600 that takes base calls of known sequences as input and generates k-mer-specific centroids. The CDSM model 600 receives encoded base calls 602 from an already base called sequence with a length of L. In one or more embodiments which will be further described in accordance with FIGS. 7 and 8, the base calls 602 can be encoded as binary permutations L×2, where 2 represents two color/intensity channels. The CDSM model 600 subdivides the encoded base calls 602 into k-mer-specific time series 612 (see step 610). Each time series represents presence or absence of a particular k-mer at each sequencing cycle in a plurality of sequencing cycles across which the base calls are generated. K-mers can be 4∧k permutations of k base positions, where 4 corresponds to four bases A, G, C and T. Accordingly, there are 4∧k permutations of k-mer-specific time series (4∧k×L×2). In accordance with step 620, the k-mer-specific time series 612 can be transformed into 4∧k permutations of transformed k-mer-specific time series 622 (4∧k×L×2). Each of the transformed time series 622 represents a predicted k-mer-specific centroid. The CDSM model 600 can correct the transformed k-mer time series 622 for context-dependent phasing effect and generate corrected k-mer-specific time series 632 (4∧k×L×2). Subsequently, the corrected k-mer time series 632 can be merged on a sequencing cycle-by-sequencing cycle basis to generate predicted per-sequencing cycle intensity values 642 (L×2). During the iterative training process, the CDSM model 600 determines a training loss (e.g., a transformation loss) by comparing the predicted per-sequencing cycle intensity values 642 against known intensity values of the encoded base calls 602 and, based on the training loss, updates the predicted k-mer-specific centroids.

In one or more embodiments, the base calls as input to the CDSM model 600 are discrete base call that are encoded as binary permutations across two color/intensity channels. FIG. 7 illustrates examples of encoded base calls at two color/intensity channels. Base C 710 has encoded base call [1, 0], representing binarized intensity value of one at the first color/intensity channel and intensity value of zero at the second color/intensity channel. Base T 720 has encoded base call [0, 1], representing binarized intensity value of zero at the first color/intensity channel and intensity value of one at the second color/intensity channel. Base G 730 has encoded base call [0, 0], representing binarized intensity value of zero at both the first and second color/intensity channels. Base A 740 has encoded base call [1, 1], representing binarized intensity value of one at both the first and second color/intensity channels.

FIG. 8 illustrates another example of encoded base calls at twenty sequencing cycles in a sequencing run. Base C is called at sequencing cycles 1, 4, 12, 13 and 17, with binarized intensities of one at the first color/intensity channel shown as white bars. Base T is called at sequencing cycles 3, 7, 10, 16, 18 and 20, with binarized intensities of one at the second color/intensity channel shown as black bars. Base A is called at sequencing cycles 2, 5, 8, 9 and 15, with binarized intensities of one at both the first and second color/intensity channels shown as diagonal-striped bars, representing the overlap between blue and white bars. Base G is called at sequencing cycles 6, 11, 14 and 19 with binarized intensities of zero at both the first and second color/intensity channels.

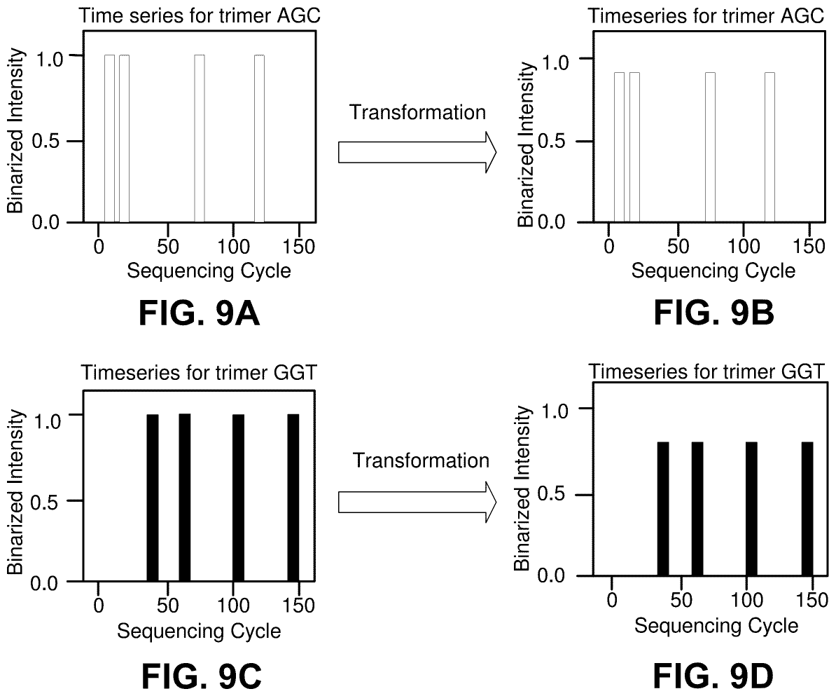

FIGS. 9A-9D illustrate examples of k-mer-specific time series and transformations thereof. FIG. 9A illustrates binarized time series for trimer AGC at 151 sequencing cycles of a sequencing run. The white bars represent binarized intensities of base C in the trimer context AGC at sequencing cycles 8, 22, 75 and 121, respectively. The binarized intensities of one are extracted from sequencing images captured at the first color/intensity channel. That is, trimer AGC is present at sequencing cycles 8, 22, 75 and 121, respectively, and is absent at remaining sequencing cycles. FIG. 9B illustrates transformed time series for trimer AGC. The white bars represent predicted centroid values corresponding to trimer AGC, which are approximately 0.85. FIG. 9C illustrates binarized time series for trimer GGT at 151 sequencing cycles of a sequencing run. The black bars represent binarized intensities of base T in the trimer context GGT at sequencing cycles 38, 62, 103 and 148, respectively. The binarized intensities of one are extracted from sequencing images captured at the second color/intensity channel. In other words, trimer GGT is present at sequencing cycles 38, 62, 103 and 148, respectively, and is absent at remaining sequencing cycles. FIG. 9D illustrates transformed time series for trimer GGT. The black bars represent predicted centroid values corresponding to trimer GGT, which are approximately 0.75.

A skilled person would appreciate that the binarized intensities as illustrated in FIGS. 9A-9D are for illustrative purposes. The binarized intensities can represent any of the bases within its corresponding k-mer context. In other words, the binarized intensities can represent base X that is to be called in the corresponding k-mer context KKX, KXK or XKK (K as known bases). Moreover, the binarized intensities of base G in the k-mer contexts may be shown as zero at both color/intensity channels and the binarized intensities of base A in the k-mer contexts may be shown as one at both color/intensity channels.

The k-mer-specific time series can be transformed using k-mer-specific transforms, such as channel mixing matrices and/or k-mer-specific phasing correction (e.g., using convolutional kernels). In one or more embodiments, the CDSM model uses k-mer-specific matrices to transform k-mer-specific time series and generate transformed time series that represent predicted k-mer-specific centroids. Each of the 4∧k time series has a corresponding k-mer-specific 2×2 matrix and after transformation, generates a corresponding predicted k-mer-specific centroid. A binarized k-mer-specific identifier can be used as a lookup index to identify the corresponding k-mer-specific matrix in order to perform the transformation. For a base X with intensity profiles captured at a given sequencing cycle and a particular k-mer-specific context, the CDSM model can transform the k-mer-specific time series by multiplying the binarized intensities of base X at the given sequencing cycle with the corresponding k-mer-specific matrix. In the alternative to a 2×2 linear transform matrix, in some cases, a 1 or c value can be added to the intensity vector to generate a 3×3 affine transform matrix. A 1 or c value can be added depending on whether an inverse or the forward transform is used. For example, [x,y] can be fed to a 2×2 matrix for a linear transform. For an affine transform, a vector [x,y, 1] or [x,y,c] is multiplied to a 3×3 matrix. The value c represents a learnable parameter through back propagation.

Consider as an example tetramer context of ATGC (where G represents the base at a current sequencing cycle). The k-mer-specific 2×2 matrix M corresponding to tetramer context ATGC is identified using a unique integer identifier computed from the k-mer itself as a lookup index. For a context length of 4, as in this example, there are 4∧4=256 possible k-mers and as a result 256 possible unique k-mer identifiers that can be used to lookup the corresponding transform matrices. The binarized intensities of base G at the current sequencing cycle is in a vector form b. Accordingly, the transformed time series with adjusted intensities i in a vector form are calculated using the following:

$$i = M \times b$$

All of the coefficients within matrix M that map binarized intensities b with adjusted intensities i are learnable through backpropagation, such that the gradient update can be applied to the entries of matrix M. When k-mer-specific 2×2 matrix M is used, the CDSM model can perform linear transformation. The CDSM model can also perform non-linear transformations. For example, the CDSM model can use k-mer-specific 3×3 matrix and perform affine transformation to generate predicted k-mer-specific centroids.

In some embodiments, the CDSM model directly learns adjusted intensities i using gradient descents. Instead of separately transforming each binarized k-mer-specific time series using a corresponding transformation matrix, the k-mer-specific centroids are learnable through backpropagation. Indeed, in some embodiments, the CDSM model treats the transformed centroids (e.g., the transformed intensities i) as learnable parameters, which can shortcut some (or all) of the computation of the transform coefficients (in matrix M) and the application through multiplying by M. For example, for each of the k-mer-specific time series, the respective binarized intensities are encoded with a dimension of k×2, where k represents the number of bases in each k-mer and 2 represents the two color/intensity channels. The CDSM model processes the binarized intensities of k-mer-specific time series as input through e.g., convolutional kernels, and generates predicted k-mer-specific centroids. In one or more embodiments, for each of the k-mer-specific time series, the respective discrete base calls are one-hot encoded with a dimension of k×4, where k represents the number of bases in each k-mer and 4 represents the four bases A, G, C and T. The CDSM model processes the one-hot encoded base calls of k-mer-specific time series as input through e.g., convolutional kernels, and generates predicted k-mer-specific centroids. The coefficients of the convolutional kernels can be optimized through backpropagation. The use of learnable k-mer-specific centroids without corresponding transformation matrices can significantly save computation power and accelerate the optimization process of k-mer-specific centroids.

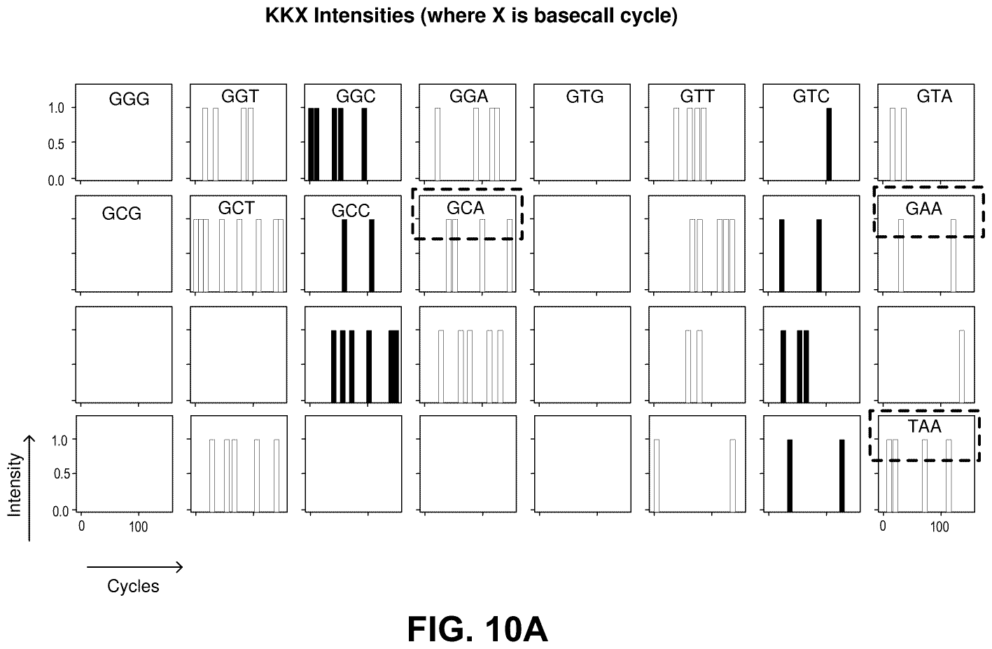

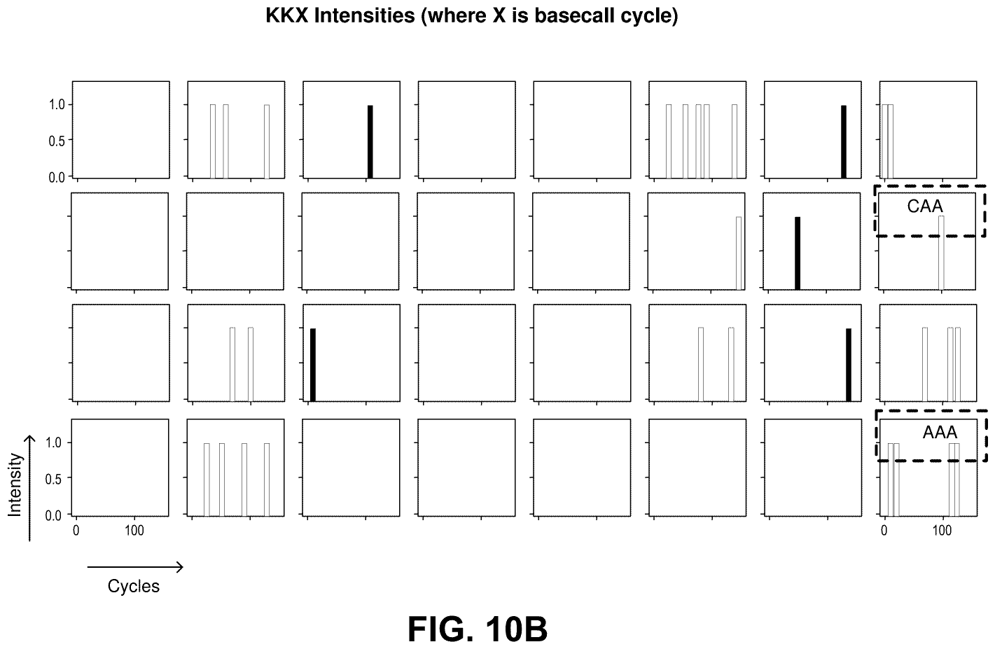

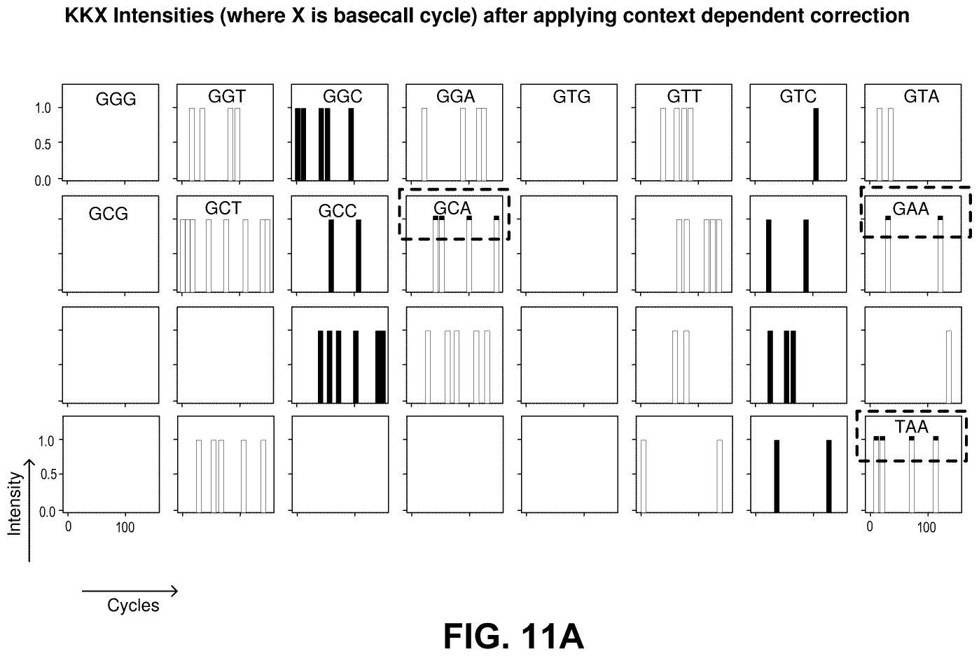

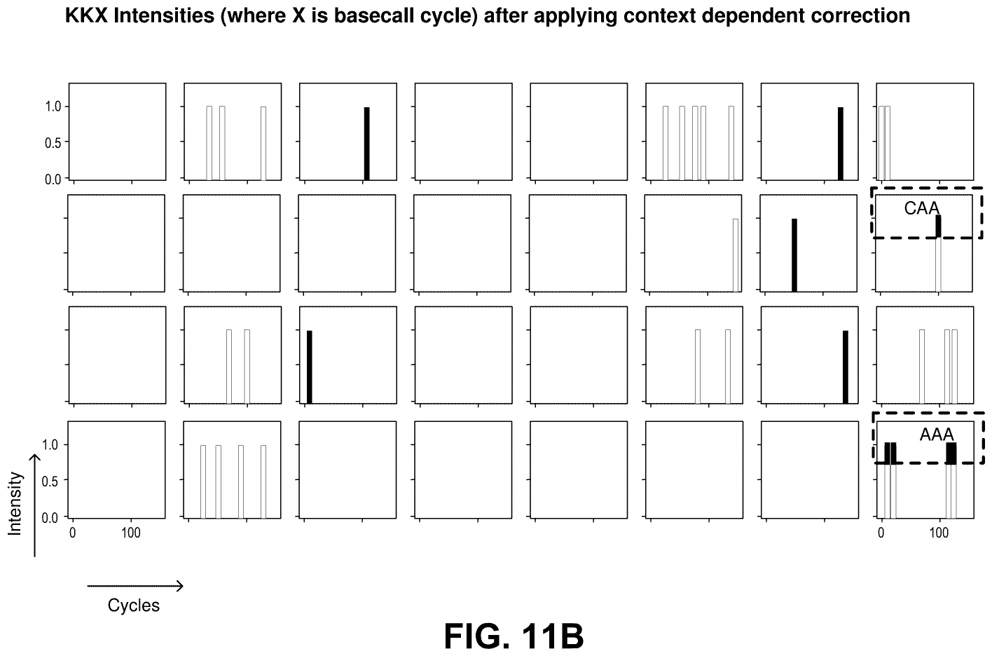

FIGS. 10A-10B and 11A-11B illustrate examples of k-mer-specific time series before and after the transformation, respectively. In particular, we are interested at trimer context KKX at 151 sequencing cycles in a sequencing run, where X is a base at a given sequencing cycle and KK are two prior bases identified at prior sequencing cycles. As described in the aforementioned embodiments and also illustrated in FIGS. 10A-10B and 11A-11B, there are sixty-four (4∧3) trimer-specific time series. The binarized intensities of each of the sixty-four time series represent presence or absence of a particular trimer at each sequencing cycle. When X is base C, the binarized intensities of trimer-specific time series AAC, ACC, AGC, ATC, CAC, CCC, CGC, CTC, GAC, GCC, GGC, GTC, TAC, TCC, TGC, TTC are captured from the first color/intensity channel. Each binarized intensity of one (shown as white bar) represents presence of the corresponding trimer at a particular sequencing cycle. When X is base T, the binarized intensities of trimer-specific time series AAT, ACT, AGT, ATT, CAT, CCT, CGT, CTT, GAT, GCT, GGT, GTT, TAT, TCT, TGT and TTT are captured from the second color/intensity channel. Each binarized intensity of one (shown as black bar) represents presence of the corresponding trimer at a particular sequencing cycle. When X is base G, the binarized intensities of trimer-specific time series AAG, ACG, AGG, ATG, CAG, CCG, CGG, CTG, GAG, GCG, GGG, GTG, TAG, TCG, TGG, TTG are minimized at both the first and second color/intensity channels. For example, the top left corner of FIG. 10A depicts the time series corresponding to trimer GGG, where the binarized intensities are minimal.

When X is base A, the binarized intensities of trimer-specific time series AAA, ACA, AGA, ATA, CAA, CCA, CGA, CTA, GAA, GCA, GGA, GTA, TAA, TCA, TGA, TTA are captured from both the first and second color/intensity channels. For example, the highlighted (e.g., outlined in boxes) time series represent the time series corresponding to GCA, GAA, TAA, CAA and AAA, respectively, shown in either FIG. 10A or 10B. It is worth noting that despite only oranges bars appear in these highlighted time series, each binarized intensity is collected at both color/intensity channels and therefore an overlap between orange and black bars.

It should also be noted that some of the trimers may not appear in a given sequence. Accordingly, their corresponding time series have minimal binarized intensities.

FIGS. 11A-11B together illustrate the sixty-four trimer-specific time series after the transformation process. Each of the sixty-four trimer-specific time series has a corresponding 2×2 matrix M. The binarized trimer-specific time series can be used as lookup indexes to identify the corresponding 2×2 matrices M. The CDSM model transforms each time series by multiplying the binarized intensities b as illustrated in FIGS. 10A and 10B, with the corresponding 2×2 matrix M to generate transformed time series with adjusted intensities i. As highlighted in FIGS. 11A and 11B, for example, five time series (GCA, GAA, TAA, CAA and AAA) show adjusted intensities i that are different from the binarized intensities b. The bottom right corner of FIGS. 10B and 11B depict the time series corresponding to AAA before and after the transformation, respectively. Trimer AAA is present at four different sequencing cycles with binarized intensities b [1, 1]. After the transformation, as shown in the bottom right corner of FIG. 11B, the transformed time series have adjusted binarized intensities i [0.8, 1.1]. The white bars represent binarized intensities of 0.8 at the first color/intensity channel and are overlapped with the black bars that represent binarized intensities of 1.1 at the second color/intensity channel.

The transformed k-mer-specific time series can further be corrected for context-based phasing. In the ideal situation of sequencing-by-synthesis (SBS) process, the lengths of all nascent strands within an analyte would be the same. Imperfections in the cyclic reversible termination (CRT) chemistry create stochastic failures that result in nascent strand length heterogeneity. In other words, the readout of the sequence copies of an analyte loses synchrony. One example is the phasing effect where an oligonucleotide in a cluster does not incorporate a nucleotide in some of the sequencing cycles and therefore, lags behind other oligonucleotides. To correct for the phasing effect, the CDSM model can apply k-mer-specific phasing coefficients to the k-mer-specific time series and generate corrected k-mer-specific time series. K-mer-specific phasing coefficients are k-mer-dependent instead of cluster-dependent. Each of the k-mer-specific time series has a corresponding k-mer-specific coefficient for phasing correction and thus, there are 4∧k permutations of k-mer-specific phasing coefficients.

In accordance with FIG. 6, the context-based phasing can be corrected after the transformation. Each of the transformed k-mer-specific time series with adjusted binarized intensities i can be corrected with the corresponding phasing coefficient to generate corrected k-mer-specific time series with corrected binarized intensities c.

In one or more embodiments in accordance with FIG. 6, the CDSM model merges the k-mer-specific time series, each representing a predicted k-mer-specific centroid, into a merged time series on a sequencing cycle-by-sequencing cycle basis. The merged time series represent predicted per-sequencing cycle intensity values, represented by the k-mer-specific centroids. For a given sequence with a length of L, for example, the discrete base calls as input to the CDSM model (L×2) can be subdivided into 4∧k time series (L×4∧k×2). After the transformation and correction for context-dependent phasing, the corrected 4∧k time series with corrected binarized intensities c can be merged to the merged time series (L×2) using e.g., a sum operator. For example, the given sequence has a length of 150 bases (L=150), which potentially has 148 trimer-based time series KKX from sequencing cycle 3 to 150. Each trimer-based time series has two known prior bases KK (K=A, G, C or T) identified at two prior sequencing cycles and a current base X (X=A, G, C or T) at a current sequencing cycle. The 150 base calls as input to the CDSM model are subdivided into 64 (4∧3) permutations of trimer-specific time series. After each permutation of time series is transformed and corrected for context-based phasing, the corrected 4∧k time series with binarized intensities c are merged to merged time series. The intensity value of each base X in a given sequencing cycle from cycle 3 to 150 in the merged time series is one of the corrected binarized intensities c corresponding to the particular trimer context KKX. As a result, the merged time series have the same dimension as the input encoded base calls, but the per-sequencing cycle intensity values in the merged time series are optimized with the correction for chemistry modulation effect caused by base context as well as context-dependent phasing.

Next, we turn to more details of the training process of the CDSM model in which the transformations parameters and context-dependent phasing coefficients are optimized.

### Training of Context-Dependent Signal Modulation (CDSM) Model

The goal of training the CDSM model is to optimize the parameters for transformations and context-dependent phasing coefficients. The model gradually combines simpler features into complex features so that the most suitable hierarchical representations can be learned from training data. Given a training dataset, the forward pass sequentially computes the output and propagates the function signals forward through the model. In the final output layer, an objective loss function measures error between the inferenced outputs and the given labels. To minimize the training error, the backward pass uses the chain rule to backpropagate error signals and compute gradients with respect to all parameters throughout the model. Finally, the parameters are updated using optimization algorithms based on stochastic gradient descent. Whereas batch gradient descent performs parameter updates for each complete dataset, stochastic gradient descent provides stochastic approximations by performing the updates for each small set of data examples. Several optimization algorithms stem from stochastic gradient descent. For example, the Adagrad, Adam and Levenberg-Marquardt training algorithms perform stochastic gradient descent while adaptively modifying learning rates based on update frequency and moments of the gradients for each parameter, respectively.

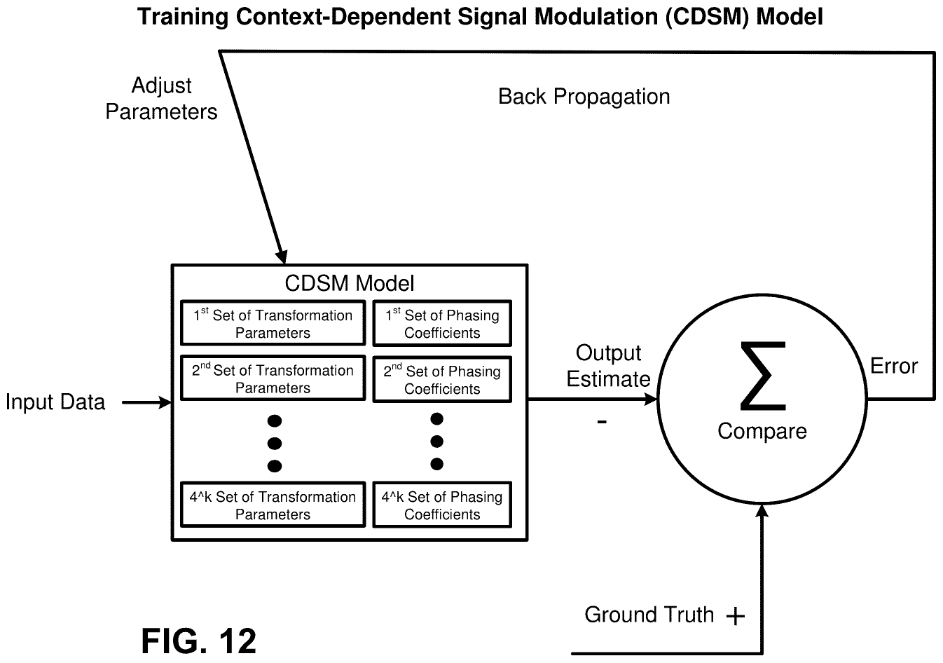

FIG. 12 illustrates a block diagram of training the CDSM model in accordance with one implementation of the technology disclosed. The CDSM model can be adjusted using back propagation based on a comparison of the output estimate and the ground truth until the output estimate progressively matches or approaches the ground truth.

In one or more embodiments, the CDSM model is trained using a plurality of already base called sequences. The number of already base called sequences as training samples can be 10-50, 50-200, 200-500, 500-1000, 1000-2000 and so on. For example, the training samples can include 512 or 1024 sequences. The base calls of these training samples as well as the corresponding intensity profiles at each sequencing cycle can be used as ground truth.

During the training process, the CDSM model receives base calls of training samples as input and subdivides them into 4∧k k-mer-specific time series. Each of the time series represents presence or absence of a particular k-mer at each sequencing cycle in a plurality of sequencing cycles across which the base calls are generated. The CDSM model transforms the k-mer-specific time series into transformed time series with adjusted binarized intensities representing predicted k-mer-specific centroids. In one or more embodiments, the transformation is performed through matrices or convolution kernels. Each of the k-mer-specific time series can have a corresponding matrix with transformation parameters that can be optimized during the training. As illustrated, the CDSM model can have a first set of transformation parameters, a second set of transformation parameters, . . . , 4∧k set of transformation parameters, each set representing the parameters of a particular k-mer-specific matrix. When 2×2 matrices are used for transformation, there are 4∧k of k-mer-specific 2×2 matrices with 4∧(k+1) learnable transformation parameters. In one or more embodiments, the k-mer-specific matrices are initialized with identity matrices, which model individual-sequence-specific behavior (or individual-k-mer-specific behavior) of k-mers. Accordingly, there are 4∧(k+2) parameters including initial parameters in the identity matrices and 4∧(k+1) learnable parameters.

The CDSM model can apply learnable k-mer-specific phasing coefficients to transformed k-mer-specific time series and generate corrected k-mer-specific time series. As illustrated, the CDSM model can have a first set of phasing parameters, a second set of phasing parameters, . . . , 4∧k set of phasing parameters, each set corresponding to a particular k-mer. These parameters can be adjusted during the training process by comparing a loss between the ground truth and the actual output. In some embodiments, the CDSM model uses a single phasing/prephasing coefficient set for all training samples.

The corrected transformed k-mer-specific time series can be merged via e.g., a sum operator into a merged time series on a sequencing cycle-by-sequencing cycle basis. The merged time series represent predicted per-sequencing cycle intensity values. The CDSM model can compare the predicted per-sequencing cycle intensity values to the ground truth intensity profiles of the training samples and determine a transformation loss based on the comparison. To update the model parameters (e.g., parameters for transformations and context-dependent phasing coefficients) with gradient descent, the use of transformation loss is to minimize the difference between the predicted per-sequencing cycle intensity values and the ground truth intensity profiles. During the backpropagation through computation graph, the gradients can flow backward through the merge step, and all of the upstreaming parameters can be updated.

An example of how gradients flow backwards through a sum operator is as follows. During backpropagation the backward pass computes the gradients with respect to the inputs of each node in the computational graph. The sum operation takes the gradients on its outputs and broadcasts it equally to all of its inputs, regardless of what the input values were during the forward pass. It follows from the fact that the local gradient for the sum operation is simply +1.0. As a result of applying the chain rule, the gradients on all inputs should be equal to the gradients on the output multiplied by 1.0 and thus, remain unchanged.

In one or more embodiments, the CDSM model iteratively fits the base calls. This process can start from a batch of sequences as training samples. For each sequence in the batch, initial respective parameters for intensity corrections (e.g., scale correction, background correction, laser ramp correction) can be estimated. The CDSM model processes discrete base calls of the batch of sequences and generates predicted k-mer-specific centroids. Via backpropagation, the CDSM model iteratively updates the parameters of the model. This iterative process can repeat e.g., 2000 times and during which, the base calls can be updated as well. For example, every thirty steps/cycles, the CDSM model, with newly updated parameters, can be inverted. That is, the CDSM model performs the base calling process by using the predicted k-mer-specific centroid and generate a finer fit for base calls. Based on the newly updated parameters and k-mer-specific centroid, the base calling system can update initial base calls to be more accurate.

In some embodiments, the base calling system uses an Adam algorithm to perform stochastic gradient descent for updating the CDSM model. The following pseudo code represents an example Adam algorithm for updating the CDSM model:

```
@jax.jit
def adam_step(dw, adam_params):
  w, moment1, moment2, t = adam_params
  w, moment1, moment2 = full(w), full(moment1), full(moment2)
  dw=full(dw)
  LEARNING_RATE = 3e−4
  e=1e−7
  delta1 = 0.9
  delta2 = 0.999
  moment1 = delta1 * moment1 + (1-delta1) * dw
  moment2 = delta2 * moment2 + (1-delta2) * dw * dw
  moment1_unbiased = moment1 / (1-delta1 ** t)
  moment2_unbiased = moment2 / (1-delta2 ** t)
  w = w − LEARNING_RATE * moment1_unbiased / (jnp.sqrt(moment2_unbiased) + e)
  t += 1
  return [w, moment1, moment2, t].
```

In particular, an exponential moving average of the gradient (dw) and the square of the gradient for each parameter (delta1 and delta2 used as ema parameters) are stored. An average normalized gradient vector can be created, and the stochastic gradient descent can be applied in the following form:

$$w = w - LEARNING\_RATE * dw\_meaned \_and \_normalized .$$

Unbiasing terms are used to debias the exp moving average at the beginning of training, these terms have no effect after a few steps (when t>>1). “e” is a small number used for numerical stability. “full( )” is used to map gradients from float 16 to float 32 for numerical stability.

With the completion of the training process, the transformation parameters and context-dependent phasing coefficients are optimized. They can be locked and thus, are no longer learnable. The predicted k-mer-specific centroids are optimized to accurately represent mean values of the intensity distributions of cluster with the same k-mer context and used for base calling unknown sequences.

### Particular Implementations of Context-Dependent Base Calling

The discussion now turns to particular implementations of context-dependent base calling, performed by the base calling pipeline disclosed herein. In one or more embodiments, the base calling system accesses current intensity data for a target cluster to be called at a current sequencing cycle of a sequencing run and context intensity data for the target cluster at preceding and/or succeeding sequencing cycles. The base context of the target cluster can be identified based on having base called the bases in previous cycles and having made preliminary base calls for future cycles. The base calling system further accesses a plurality of k-mer-specific centroids stored in the memory and determines respective k-mer-specific centroids that correspond to the base context of the target cluster at the current sequencing cycle. By comparing the respective k-mer-specific centroids with the current intensity data, the base calling system determines the base call of the target cluster.

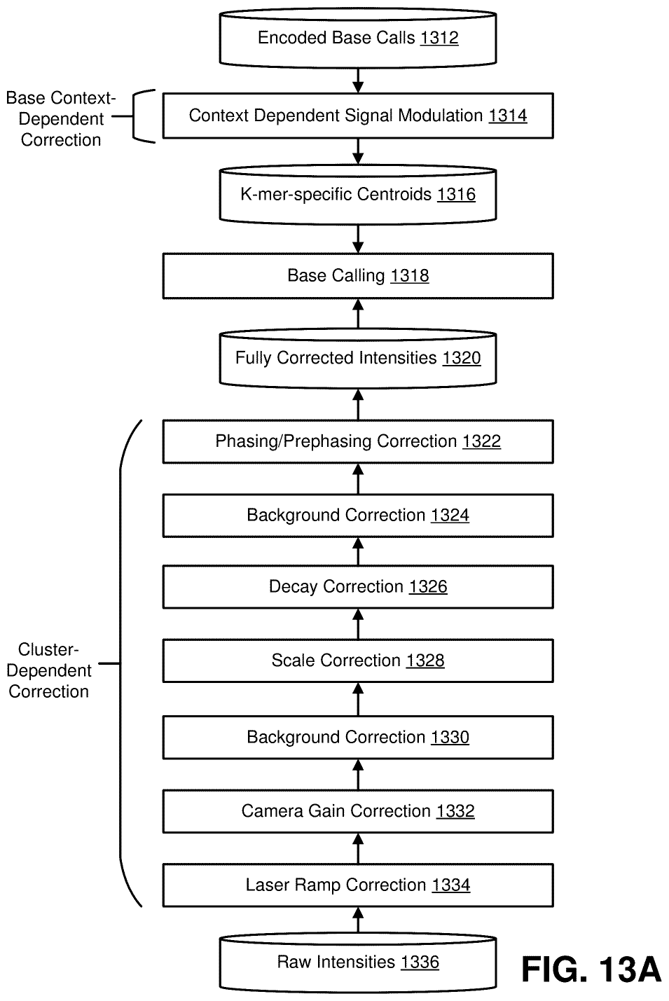

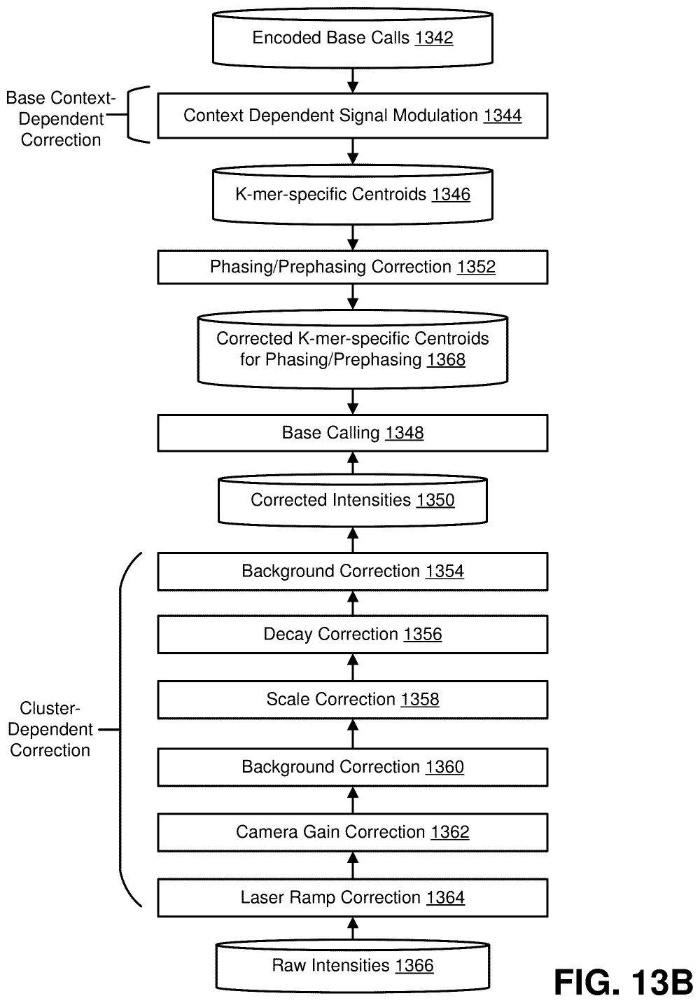

FIGS. 13A and 13B illustrate two examples of generating predicted k-mer-specific centroids via the CDSM model and using the k-mer-specific centroids for base calling. The context-dependent base calling illustrated in FIG. 13B shares a majority of the steps in FIG. 13A. However, FIG. 13B differs from FIG. 13A in the workflow as to when the phasing/prephasing correction is performed.

Using maximum likelihood sequence estimation (MLSE), the base calling system identifies or determines a base call without inverting phasing. Phasing correction gathers the signal into a single cycle and enables an easier way to make base calls by comparing intensities on a per-cycle basis (e.g., after phasing correction the base calling system only looks at the intensities from cycle n to determine a base call). In some cases, the drawback of phasing correction is that it amplifies noise, and thus, after phasing correction, the intensities from clusters detected by the base calling system might exhibit relatively higher variation and thereby cause a base call error. Once a base call error is made, this error is propagated to neighboring cycles through the decision feedback loop of base calling. Such an error-propagated decision feedback loop can result in incorrect context and the wrong centroid to generate a base call for the following cycles. In some cases, a wrong base call might throw off (or otherwise adversely reconfigure) the RTA channel estimation algorithm and provoke more errors.

To overcome these issues using MLSE, the base calling system identifies a sequence of k bases that can explain the shape of the signal without performing phasing correction. Thus, the chance for decision feedback errors is reduced. The base call decision is made by matching the signal along more than 1 cycles of intensities. In some cases, the base calling system uses a multi-k-mer approach (or a brute-force approach 0 by determining signals based on all possible 3-mers or 5-mers and selecting the k-mer that causes the signal to be closest to the observed signal. The base calling system further runs these candidate sequence calls through the CDSM model (in the forward direction) and compares 3 or 5 cycles worth of intensities. (As indicated above, when feeding candidate sequence calls through the CDSM model, the base calling system also applies sequence dependent effects and a forward version of phasing.) In some embodiments, the base calling system applies such a multi-k-mer approach followed by candidate sequence calls at every cycle, while in other embodiments the base calling system applies more sophisticated algorithms based on the fact that once the problem is solved for a 5-mer, shifting one cycle to the right will result in redundant computations. This every cycle multi-k-mer approach is akin to a tree search algorithm where a system executes all possible branches of the tree each corresponding to a different sequence. In some embodiments, the base calling system uses an algorithm based on dynamic programming (e.g., a Viterbi algorithm, which is the core of the MLSE algorithm). In certain cases, the base calling system uses shortcut techniques with hardware acceleration to precompute all the sequence permutations and parallelize the matching to the data using parallel computations.

As illustrated in both figures, the CDSM model is trained to take as input encoded base calls 1312/1342 of already base called sequences and performs context-dependent signal modulation 1314/1344. The CDSM model iteratively learns k-mer-specific centroids 1316/1346, which can be stored in memory for base calling.

When a target cluster immobilized in a flow cell is to be base called, the sequencing platform generates raw intensities 1336/1366 of the target cluster, referring to the raw signals captured by the sequencing platform. In some embodiments, the raw intensities 1336/1366 are further corrected to generate corrected intensities, e.g., fully corrected intensities 1320 as illustrated in FIG. 13A and corrected intensities 1350 as illustrated in FIG. 13B. Examples of raw intensity corrections can include laser ramp correction 1334/1364, camera gain correction 1332/1362, background corrections 1330/1360 and 1324/1354, scale correction 1328/1358, decay correction 1326/1356 and phasing/prephasing correction 1322/1352. Examples of background correction, decay correction and phasing/prephasing correction are described in U.S. Pat. No. 11,423,306 and U.S. Patent Publication No. US2020/0364565A1. In some cases, using the two different background corrections 1330/1360 and 1324/1354 help achieve a better fit to the data.

In particular, the phasing/prephasing correction 1322/1352 is to address loss of synchrony in the readout of the sequence copies of an analyte loses synchrony caused by phasing and prephasing. Phasing is caused by incomplete removal of 3′ terminators and fluorophores as well as sequences in the analyte missing an incorporation cycle. Prephasing is caused by the incorporation of nucleotides without effective 3′-blocking. Incomplete extension due to phasing results in lagging strands (e.g., t−1 from the current cycle). Addition of multiple nucleotides or probes in a population of identical strands due to prephasing results in leading strands (e.g., t+1 from the current cycle). Phasing and prephasing effects are nonstationary distortions and thus the proportion of sequences in each analyte that is affected by phasing and prephasing increases with cycle number, which hampers correct base identification and limiting the length of useful sequence reads.

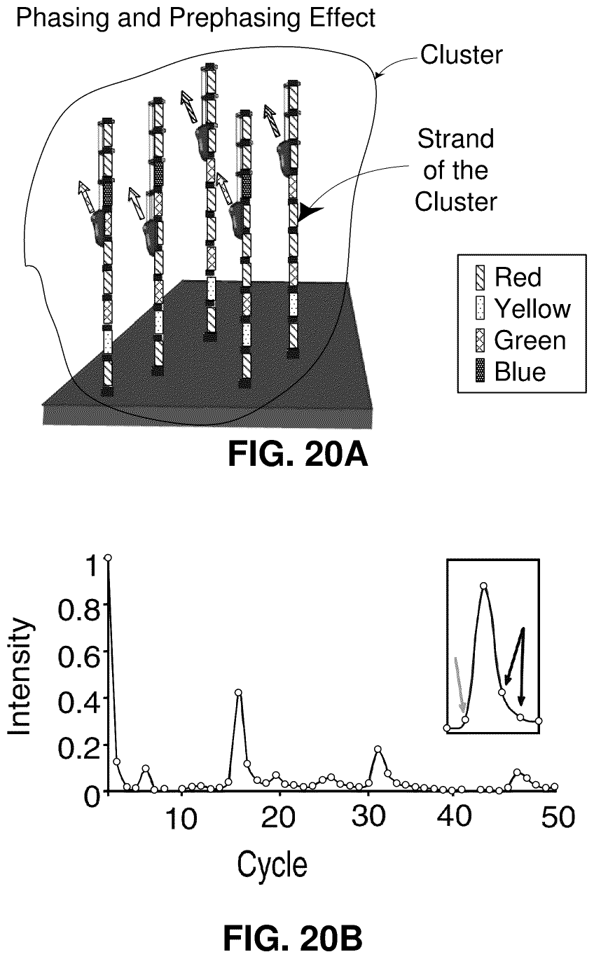

FIGS. 20A and 20B illustrate an example of the phasing and prephasing effects. FIG. 20A shows that some strands of an analyte lead (red) while others lag behind (blue), leading to a mixed signal readout of the analyte. FIG. 20B depicts the intensity output of analyte fragments with “C” impulses every 15 cycles in a heterogeneous background. Notice the anticipatory signals (gray arrow) and memory signals (black arrows) due to the phasing and prephasing effect.

The decay correction 1326/1356 is to address the signal decay, for example, fading of the intensities of the fluorophores that are incorporated into the template sequences during the sequencing-by-synthesis process. As sequencing proceeds, accurate base calling becomes increasingly difficult, because signal strength decreases and noise increases, resulting in a substantially decreased signal-to-noise ratio. It has been observed that later synthesis steps attach tags in a different position relative to the sensor than earlier synthesis steps. When the sensor is below a sequence that is being synthesized, signal decay results from attaching tags to strands further away from the sensor in later sequencing steps than in earlier steps. This causes signal decay with progression of sequencing cycles.

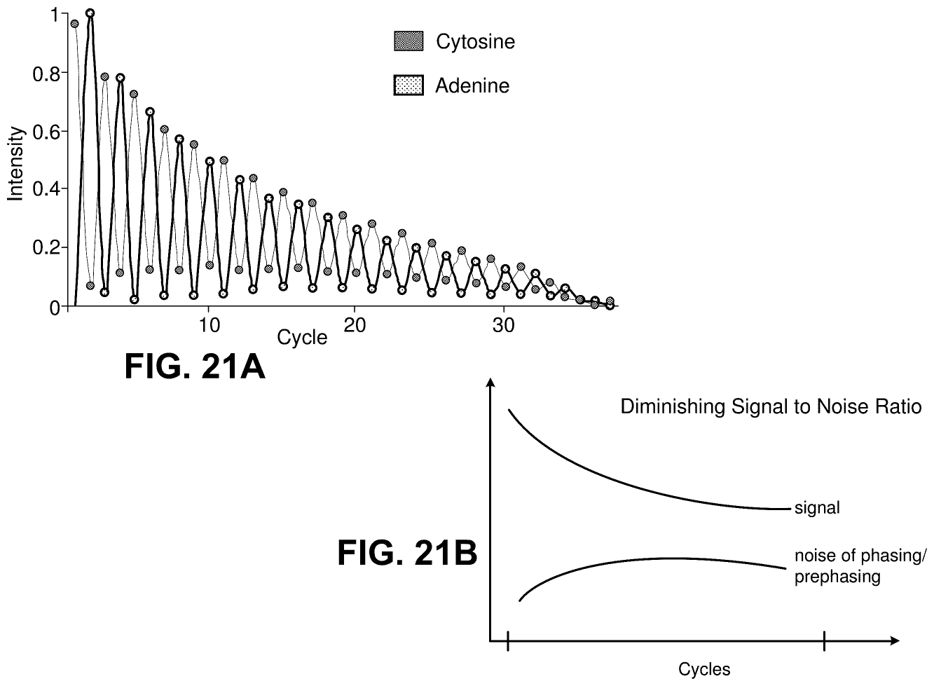

FIG. 21A illustrates an example of fading (also called dimming or signal decay), in which signal intensity is decreased as a function of cycle number in a sequencing run of a base calling operation. Fading is an exponential decay in fluorescent signal intensity as a function of base calling cycle number. As the sequencing run progresses, the analyte strands are washed excessively, exposed to laser emissions that create reactive species, and subjected to harsh environmental conditions. All of these lead to a gradual loss of fragments in each analyte, decreasing its fluorescent signal intensity. As illustrated, the intensity values of analyte fragments with AC microsatellites (simple sequence tandem repeats of cytosine and adenine) show exponential decay. FIG. 21B conceptually illustrates a decreasing signal-to-noise ratio as cycles of sequencing progress. For example, as sequencing proceeds, accurate base calling becomes increasingly difficult, because signal strength decreases and noise increases, resulting in a substantially decreased signal-to-noise ratio.

The background corrections 1330/1360 and 1324/1354 are to address background variation. Background intensity of a particular sensor is relatively steady between cycles, but varies across the sensors. Positioning of the illumination source, which can vary by illumination color, creates a spatial pattern of background variation over a field of the sensors. It has been found that manufacturing differences among the sensors were observed to produce different background intensity readouts, even between adjoining sensors. In a first approximation, idiosyncratic variation among sensors can be ignored. In a refinement, the idiosyncratic variation in background intensity among sensors can be taken into account. Background intensity can be a constant parameter to be fit, either overall or per pixel. Alternatively, different background intensities are taken into account and corrected accordingly.

The scale correction 1328/1358 is to address the variations in the intensities of clusters. When clusters are immobilized on the surface of the flow, their size and shape may vary. A larger-sized cluster includes more template oligonucleotides than a small-sized cluster and thus, may show higher intensity values when more fluorophores are incorporated into the oligonucleotides. The scale correction 1328/1358 can account for the difference in the scale of the intensities of clusters.

In some embodiments, at least one of the camera gain correction 1332/1362, background correction 1330/1360 and 1324/1354, scale correction 1328/1358, decay correction 1326/1356 and phasing/rephrasing correction 1322/1352 can be iteratively learned by training the base calling system. Each of the correction processes can involve learnable and cluster-dependent parameters, that is, each cluster or a batch of clusters can have a particular set of learnable parameters used to correct for inter-cluster intensity variations. During the training process of the base calling system where these parameters are iteratively optimized, the transformation parameters and context-dependent phasing parameters in the CDSM models can be locked. In other words, the base calling system does not learn the chemistry effects caused by base context but leverages the optimized transformation parameters and context-dependent phasing parameters.

It should also be noted that the orders of signal corrections as illustrated in FIGS. 13A and 13B are for illustrative purposes. The order can be adjusted without narrowing the scope of the technology disclosed.

As further illustrated in FIG. 13A, the raw signals 1336 for the target cluster to be called at a current sequencing cycle N are corrected for laser ramp (1334), camera gain (1332), background (1330 and 1324), scale (1328) and decay (1326). Therefore, the current intensity data used by the base calling system to base call the cluster is the fully corrected intensities 1320. Similarly, the base context data at prior and/or succeeding sequencing cycles is the fully corrected intensities used to call the context bases (i.e., prior and/or succeeding bases). The base calling system can access k-mer-specific centroids 1316 and select the respective centroids that correspond to the k-mer context of the target cluster. By comparing the respective k-mer-specific centroids with the current intensity data, the base calling system can base call the cluster (see 1318).

In some embodiments, the current intensity data of the target cluster at a current sequencing cycle N is processed using inverse matrices for base calling. In particular, the current intensity data (i.e., fully corrected intensities 1320) can be expressed as 1×2 array fci(c). In certain embodiments, “fci” refers to fully corrected intensities 1320 and/or to application of the per cluster corrections learned in the CDSM model (e.g., scale, offset, decay, and camera gain). In some cases, the base calling system can base call at different stages in the CDSM model by carrying the intensities from the instrument (e.g., the transformed signal) output backwards through the CDSM model inverse and provide the output to a given stage. The base calling system can then iterate over the 4 possible base calls given the context and carry this forward to the same model stage to find the base call that produces the least difference with the transformed signal coming from the instrument.

The target cluster has two prior base calls identified at prior sequencing cycles N−2 and N−1. Given the particular base context, the base calling system selects respective matrices Sk that correspond to the base context.

For each of the respective matrices Sk, the base calling system computes the binarized base calls bc(c) by multiplying the inverse matrix Sk with the current intensity data as follows:

$$bc ( c ) = invert ( matrix Sk ) * fci ( c )$$

Next, the base calling system calculates a normalized difference x(c) between the binarized base calls and rounded binarized base calls as follows:

$$x ( c ) = norm ( bc ( c ) - round ( bc ( c ) ) )$$

The binarized base call bc(c) that produces the lowest value of x(c) is determined as the base call for the target cluster.

For a k-mer that has a base to be called at sequencing cycle N, when N<k, the base context (e.g., number of prior base calls) of the target cluster can be insufficient to determine which of the four k-mer-specific centroids 1316 should be selected to compare with the fully corrected intensities 1320 of the target cluster. The base calling system can compare each of the k-mer-specific centroids 1316 with the current intensity data of the target cluster and determine which centroid fits the best.

Consider an example of base calling the target cluster at sequencing cycle 1 (N=1). No prior base context of the target cluster can be determined because this is the first cycle. The k-mer-specific centroids 1316 include sixty-four trimer-specific centroids (k=3). The base calling pipeline can compare each of the sixty-four centroids with the fully corrected intensities 1320 of the target cluster for base calling.

At sequencing cycle 2 (N=2), now the target cluster has a base context including a known prior base call (e.g., base A) identified at sequencing cycle 1. Therefore, the base calling pipeline does not need to compare all of the sixty-four trimer-specific centroids with the current intensity data of the target cluster. Instead, sixteen trimer-specific centroids with base A as the first base AGG, AGT, AGC, AGA, ACG, ACT, ACC, ACA, AAG, AAT, AAC, AAA, ATG, ATT, ATC and ATA can be selected. These trimer-specific centroids are used to call the target cluster at sequencing number 2.

When N≥k, the target cluster has a base context including more prior base calls identified at sequencing cycles N−1, N−2, . . . , 1. Only four k-mer-specific centroids are needed to compare to the intensity profiles of the cluster to be called. For example, when k=3 and N≥3, the base context can include two known prior base calls that are identified at sequencing cycles N−2 and N−1. The base calling pipeline can compare four of the trimer-specific centroids with the same base context with the current intensity data for base calling the target.

As illustrated in FIG. 13B, the raw intensities 1366 are corrected for laser ramp (1364), camera gain (1362), background (1360 and 1354), scale (1358) and decay (1356). But unlike FIG. 13A where the raw intensities 1336 are corrected for phasing/prephasing before generating fully corrected intensities 1320, in FIG. 13B, the corrected intensities 1350 are not corrected for phasing/prephasing effect. Instead, the k-mer-specific centroids 1346 are corrected for phasing/prephasing effect (see 1352). The base calling system then compares the corrected k-mer-specific centroids 1346 with the corrected intensities 1350 to base call the target cluster.

### Objective Indicia of Inventiveness and Non-Obviousness

FIG. 14 illustrates the comparison between predicted intensities generated by the base calling pipeline and observed intensities extracted from the sequencing images at each sequencing cycle. For the sake of simplicity, the intensities at the first color/intensity channel are compared. As described in the aforementioned embodiments, the predicted intensities can be k-mer-specific centroids that are learned by training the base calling pipeline. In a sequencing run of approximately 150 sequencing cycles, the predicted intensities (blue color) are correlated with the observed intensities (orange color). As highlighted in the circle, the sequence that is base called has repeated bases with similar intensities at successive sequencing cycles. The predicted intensities are well correlated with the observed signals with minimal discrepancy.

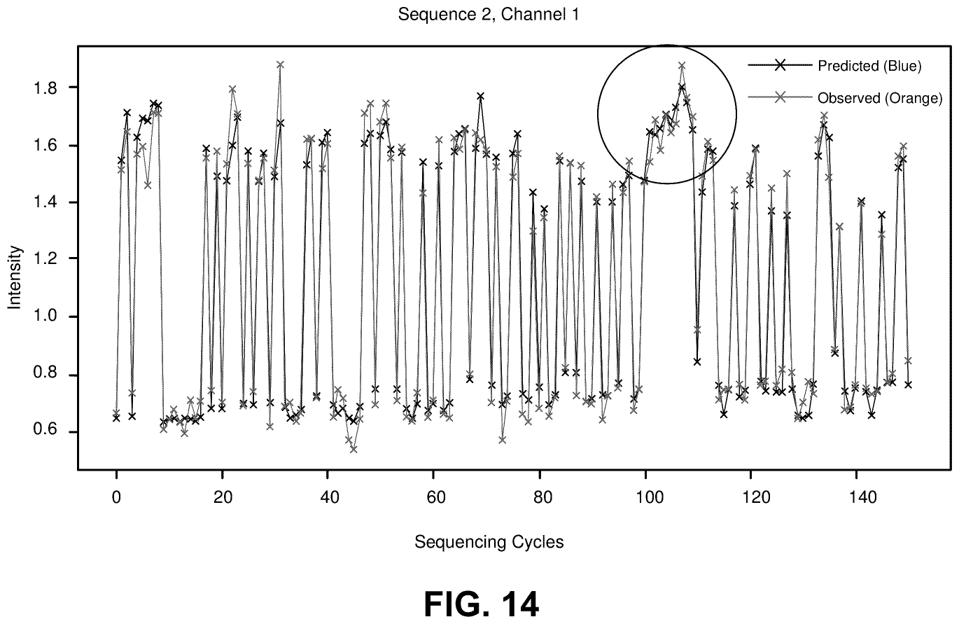

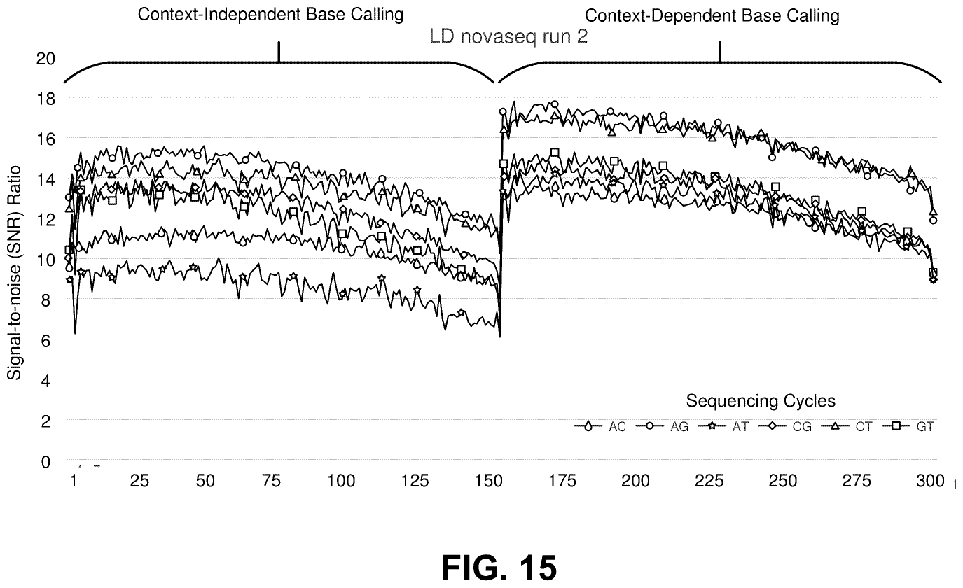

FIG. 15 illustrates comparisons in signal-to-noise (SNR) ratios of context-independent base calling and context-dependent base calling over a plurality of sequencing cycles of a sequencing run. Specifically, the comparison shows the same run cycles analyzed using two different methods. The first method shows context-independent base calling performed at sequencing cycles 1-151, with a range of SNR ratio from 6 to 16. The second method shows context-dependent base calling performed at sequencing cycles 1-151 as described in the aforementioned embodiments. As illustrated, the context-dependent base calling improves the SNR ratio to a range of 9 to 18.

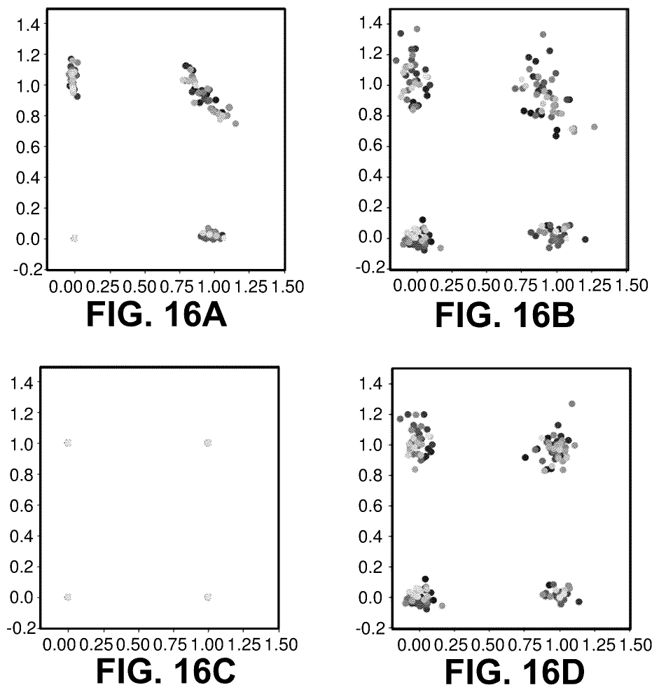

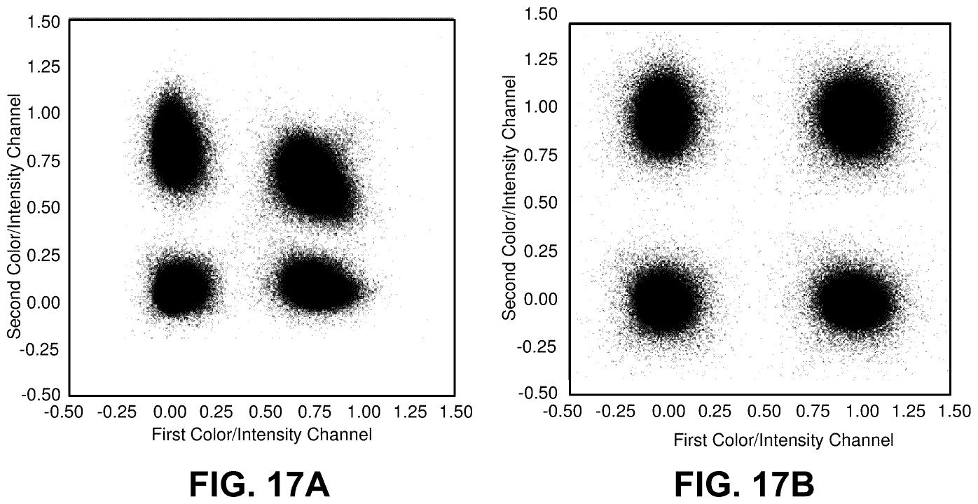

FIGS. 16A-16D illustrate comparison between intensity distribution of a single cluster without and with corrections for context-dependent effects over a plurality of sequencing cycles of a sequencing run. As shown in FIGS. 16A-16D, the first row is model output, the second row is model input. The left column is a simulated sequence through the model showing that the characteristic spread of the clouds has been properly captured in the model, and the right column shows an actual signal from the sequencer (top) and its sequence dependent modulated correction (bottom). FIG. 16A illustrates the intensity distribution of simulated signals for context-independent base calling. FIG. 16B illustrates the intensity distribution of observed intensities extracted from sequencing images captured from the first and second color/intensity channels. In accordance with FIGS. 17A, the observed intensities here can be fully corrected intensities that are corrected for laser ramp, camera gain, background, scale and decay effects. When base context of the cluster is not taken into consideration, for both simulated signals and observed signals, the shapes and dimensions of the intensity distributions vary. For example, the intensity distributions of simulated signals for base A, as illustrated in FIG. 16A, vary from 0.75 to 1.25 and from 0.8 to 1.2 at the at the first and second color/intensity channel, respectively. Similarly, the observed intensities for base A, as illustrated in FIG. 16B, vary from 0.75 to 1.25 and from 0.6 to 1.4 at the first and second channels, respectively. Thus, during the base calling, for each of the four intensity distributions corresponding to bases A, G, C and T, it is important to use both the corresponding centroid and covariance of the distribution to fit to the intensities of the cluster. Moreover, the variations in the intensity distributions caused by base context may cause miscalls, especially when an intensity profile of a target cluster to be base called is close to a decision boundary, i.e., between two intensity distributions of different bases, for example, base A and base C, base A and base T.

FIG. 16C illustrates the intensity distributions of simulated signals for context-dependent base calling. FIG. 16D illustrates the intensity distributions of observed intensities extracted from sequencing images captured from the first and second color/intensity channels and corrected for context-dependence. When base context of the cluster is taken into consideration for each sequencing cycle, the corrected intensities have a uniform distribution for each of the four bases A, G, C and T. The observed signals, as illustrated in FIG. 16D, also show improvement in the intensity distributions of the four bases. Compared to FIG. 16B, for example, bases A and T show almost circular distributions. The context-dependent intensity correction reduces the error rate of base calling, because each intensity distribution has a substantially uniform shape and dimension. Moreover, because each centroid is corrected to accurately represent a mean value of the intensities of bases with the same context, the covariance of the distribution may not be needed to base call a cluster, which saves computation power.

FIGS. 17A and 17B illustrate comparisons between the intensity distribution of a plurality of clusters without and with correction for context-dependent effects. Unlike FIGS. 16A-16D where the intensity distributions are either simulated or measured for a single cluster, here, the intensities are acquired from 2,048 clusters. FIG. 17A illustrates the intensity distribution of the fully corrected intensities of the clusters without correction for context-dependent effects. Consistent with FIGS. 16A and 16B, the dimensions of the distributions for four bases vary. Ideally, in a two-channel sequencing system, the centroids of the distributions of four bases A, C, T and G should be located at normalized intensities of (1, 1), (1, 0), (0, 1) and (0, 0) at the two color/intensity channels, respectively. As illustrated in FIG. 17A, the centroids of the distributions of four bases A, C, T and G are located at normalized intensities of (0.75, 0.72), (0.75, 0.1), (0.1, 0.75) and (0.1, 0.1) at the two color/intensity channels. The intensity distributions of bases A and C are in proximity to one another, increasing the risk of miscalls. FIG. 17B illustrates the intensities distributions of clusters with correction for context-dependent effects. The shapes and dimensions of each distribution is substantially uniform. The centroids of the distributions of four bases A, C, T and G are at normalized intensities of (1, 1), (1, 0), (0, 1) and (0, 0) at two color-intensity channels, respectively.

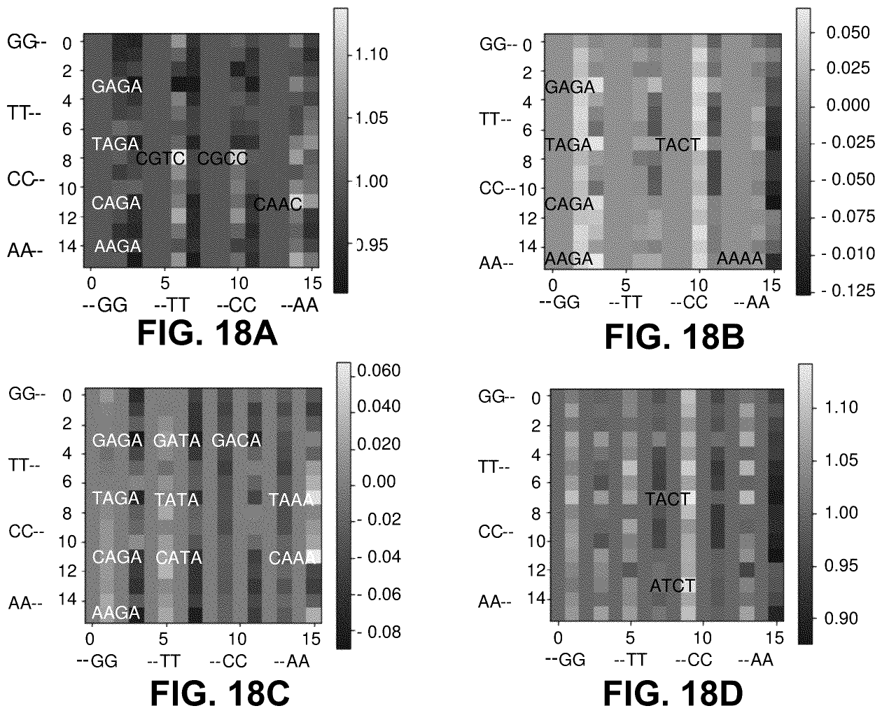

FIGS. 18A-18D illustrate examples of tetramer-specific matrices that transform tetramer-specific time series to predicted tetramer-specific centroids. Here, the tetramer-specific context KKKX represents a base X that is to be called at a current sequencing cycle N with three prior bases KKK that are identified at prior sequencing cycles N−3, N−2 and N−1. Accordingly, there are 256 (4∧4) permutations of base positions, including GGGG, GGGA, GGGC, GGGT, GGAG, . . . , AAAA. Each of the FIGS. 18A-18D illustrates 256 of 2×2 matrices. As described in the aforementioned embodiments, each of the 256 tetramer-specific time series can be transformed using a corresponding 2×2 matrix with learnable transformation parameters. The matrices are initialized as identity matrices

$$[ \begin{matrix}1 & 0 \\ 0 & 1\end{matrix} ] .$$

of The transformation parameters in these tetramer-specific matrices can be optimized during iterative training of the base calling pipeline via backpropagation. The color bars in FIGS. 18A-18D indicate the deviations of transformation parameters from the initial identity matrices. As illustrated in FIG. 18A, tetramers CGTC, CGCC and CAAC show significant positive deviation from the initial intensity of one, and tetramers GAGA, TAGA, CAGA and AAGA show negative deviation. Similarly in FIG. 18B, tetramers GAGA, TAGA, CAGA, AAGA and TACT show positive deviation from the intensity of zero, while tetramer AAAA shows negative deviation. In FIG. 18C, tetramers GAGA, TAGA, CAGA, AAGA, GATA, TATA, CATA and GACA show negative deviation from the intensity of zero, while tetramers TAAA and CAAA shows positive deviation from the intensity of zero. In FIG. 18D, tetramers TACT and ATCT show positive deviation from the intensity of one.

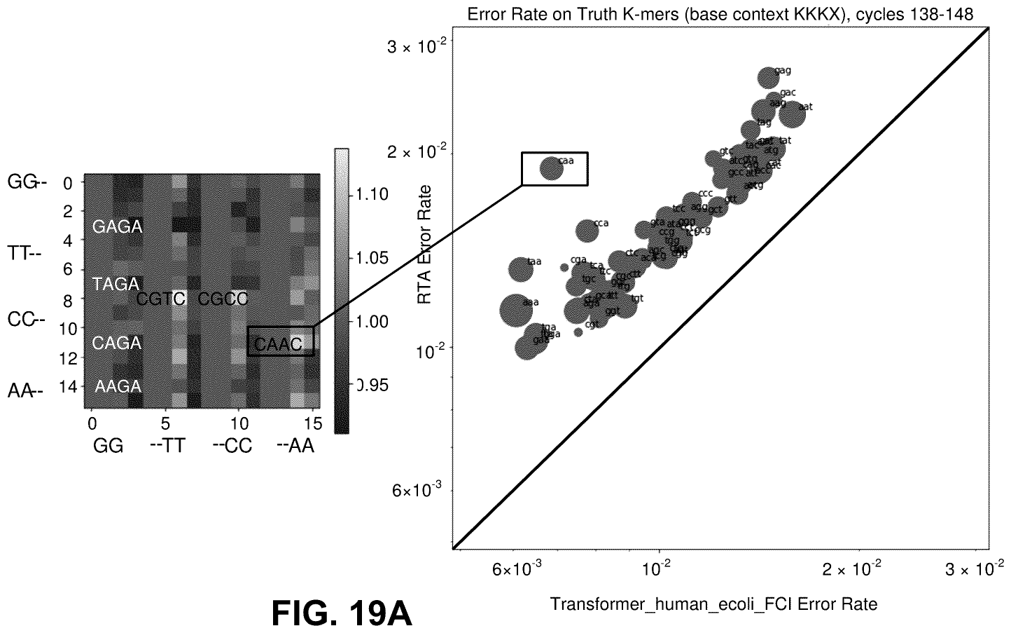

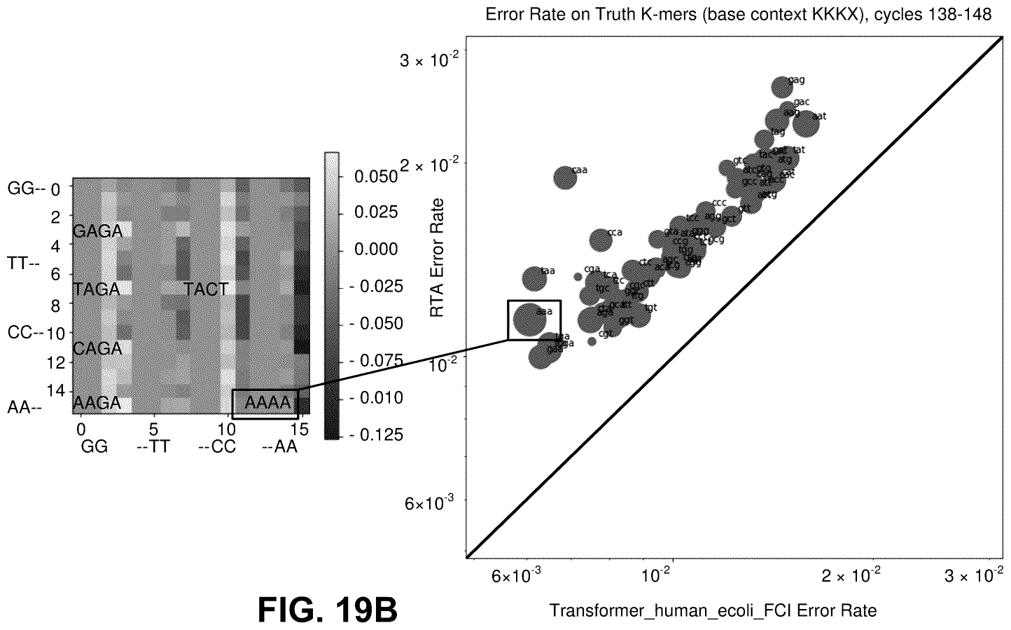

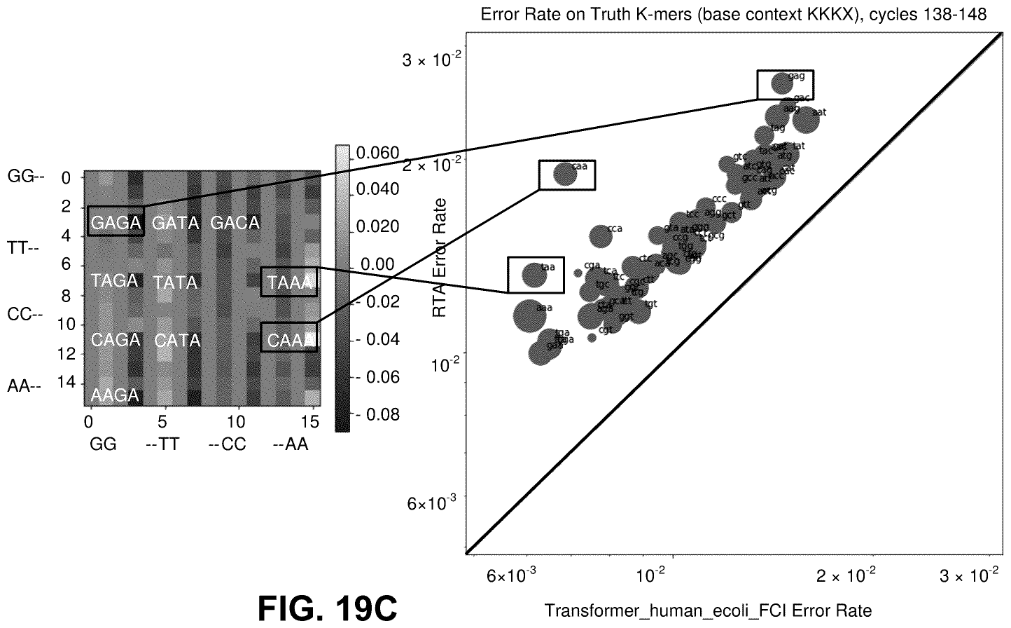

The identification of the tetramers with transformation matrices significantly deviated from identity matrices sheds light on the study of the intensity variations caused by chemistry modulation effect. FIGS. 19A-19C depict the correlations between identified tetramers and corresponding error rates when base calling clusters using an independent base caller (e.g., “attentionRTA” or “Transformer”) with the identified tetramer context. FIG. 19A illustrates the correction of the identified tetramers as illustrated in FIG. 18A with the observed error rate when base calling clusters with the identified tetramer context. The observed error rate is categorized by base context KKKX, where X is the base to be called at a given sequencing cycle and KKK are three prior bases identified at prior sequencing cycles. Each data point in blue circular form represents error rate of clusters with a particular base context. The transformation matrix corresponding to highlighted tetramer CAAC is determined to have significant positive deviation from the identity matrices, which is consistent with the high error rate of clusters with three prior bases CAA. FIG. 19B illustrates the correction of the identified tetramers as illustrated in FIG. 18B with the observed error rate when base calling clusters with the identified tetramer context. The transformation matrix corresponding to highlighted tetramer AAAA is determined to have significant negative deviation from the identity matrices, which is consistent with the error rate of clusters with three prior bases AAA. Similarly, FIG. 19C illustrates the correction of the identified tetramers as illustrated in FIG. 18C with the observed error rate for base calling. The transformation matrices corresponding to highlighted tetramer TAAA and CAAA is determined to have significant positive deviations from the identity matrices, consistent with the high error rate of clusters with three prior bases TAA and CAA, respectively. The transformation matrix corresponding to highlighted tetramer GAGA shows deviation from the identity matrices, consistent with the error rate of clusters with three prior bases GAG. These correlations also validate the chemistry modulation effect caused by base context, especially when prior bases in the context include one or more base A.

### Computer System

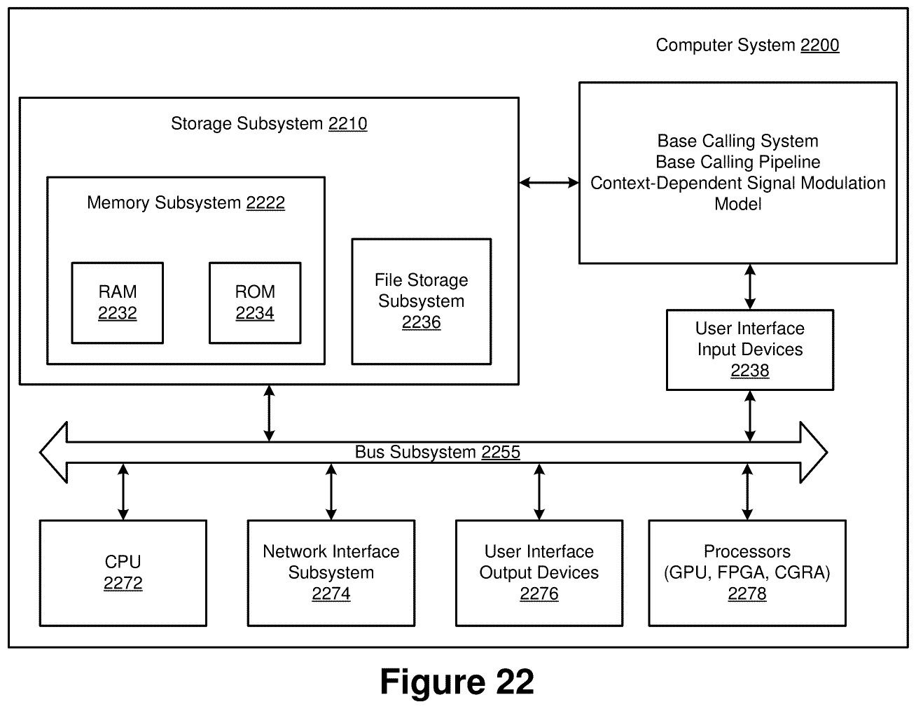

FIG. 22 is a computer system 2200 that can be used to implement the technology disclosed. Computer system 2200 includes at least one central processing unit (CPU) 2272 that communicates with a number of peripheral devices via bus subsystem 2255. These peripheral devices can include a storage subsystem 2210 including, for example, memory devices and a file storage subsystem 2236, user interface input devices 2238, user interface output devices 2276, and a network interface subsystem 2274. The input and output devices allow user interaction with computer system 2200. Network interface subsystem 2274 provides an interface to outside networks, including an interface to corresponding interface devices in other computer systems.

In one implementation, at least one of the base calling system, base calling pipeline or Context-Dependent Signal Modulation (CDSM) model is communicably linked to the storage subsystem 2210 and the user interface input devices 2238.

User interface input devices 2238 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system 2200.

User interface output devices 2276 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system 2200 to the user or to another machine or computer system.

Storage subsystem 2210 stores programming and data constructs that provide the functionality of some or all of the modules and methods described herein. These software modules are generally executed by processors 2278.

Processors 2278 can be graphics processing units (GPUs), field-programmable gate arrays (FPGAs), application-specific integrated circuits (ASICs), and/or coarse-grained reconfigurable architectures (CGRAs). Processors 2278 can be hosted by a deep learning cloud platform such as Google Cloud Platform™, Xilinx™, and Cirrascale™. Examples of processors 2278 include Google's Tensor Processing Unit (TPU)™, rackmount solutions like GX4 Rackmount Series™, GX15 Rackmount Series™, NVIDIA DGX-1™, Microsoft′ Stratix V FPGA™, Graphcore's Intelligent Processor Unit (IPU)™, Qualcomm's Zeroth Platform™ with Snapdragon processors™, NVIDIA's Volta™, NVIDIA's DRIVE PX™, NVIDIA's JETSON TX1/TX2 MODULE™, Intel's Nirvana™, Movidius VPU™, Fujitsu DPI™, ARM's DynamicIQ™, IBM TrueNorth™, Lambda GPU Server with Testa V100s™, and others.

Memory subsystem 2222 used in the storage subsystem 2210 can include a number of memories including a main random access memory (RAM) 2232 for storage of instructions and data during program execution and a read only memory (ROM) 2234 in which fixed instructions are stored. A file storage subsystem 2236 can provide persistent storage for program and data files, and can include a hard disk drive, a floppy disk drive along with associated removable media, a CD-ROM drive, an optical drive, or removable media cartridges. The modules implementing the functionality of some implementations can be stored by file storage subsystem 2236 in the storage subsystem 2210, or in other machines accessible by the processor.

Bus subsystem 2255 provides a mechanism for letting the various components and subsystems of computer system 2200 communicate with each other as intended. Although bus subsystem 2255 is shown schematically as a single bus, alternative implementations of the bus subsystem can use multiple busses.

Computer system 2200 itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system 2200 depicted in FIG. 22 is intended only as a specific example for purposes of illustrating the preferred implementations of the present invention. Many other configurations of computer system 2200 are possible having more or less components than the computer system depicted in FIG. 22.

Each of the processors or modules discussed herein may include an algorithm (e.g., instructions stored on a tangible and/or non-transitory computer readable storage medium) or sub-algorithms to perform particular processes. The base calling pipeline can be implemented utilizing any combination of dedicated hardware boards, DSPs, processors, etc. Alternatively, the base calling pipeline implemented utilizing an off-the-shelf PC with a single processor or multiple processors, with the functional operations distributed between the processors. As a further option, the modules described below may be implemented utilizing a hybrid configuration in which some modular functions are performed utilizing dedicated hardware, while the remaining modular functions are performed utilizing an off-the-shelf PC and the like. The modules also may be implemented as software modules within a processing unit.

Various processes and steps of the methods set forth herein can be carried out using a computer. The computer can include a processor that is part of a detection device, networked with a detection device used to obtain the data that is processed by the computer or separate from the detection device. In some implementations, information (e.g., image data) may be transmitted between components of a system disclosed herein directly or via a computer network. A local area network (LAN) or wide area network (WAN) may be a corporate computing network, including access to the Internet, to which computers and computing devices comprising the system are connected. In one implementation, the LAN conforms to the transmission control protocol/internet protocol (TCP/IP) industry standard. In some instances, the information (e.g., image data) is input to a system disclosed herein via an input device (e.g., disk drive, compact disk player, USB port etc.). In some instances, the information is received by loading the information, e.g., from a storage device such as a disk or flash drive.

A processor that is used to run an algorithm or other process set forth herein may comprise a microprocessor. The microprocessor may be any conventional general purpose single- or multi-chip microprocessor such as a Pentium™ processor made by Intel Corporation. A particularly useful computer can utilize an Intel Ivybridge dual-12 core processor, LSI raid controller, having 128 GB of RAM, and 2 TB solid state disk drive. In addition, the processor may comprise any conventional special purpose processor such as a digital signal processor or a graphics processor. The processor typically has conventional address lines, conventional data lines, and one or more conventional control lines.

The implementations disclosed herein may be implemented as a method, apparatus, system or article of manufacture using standard programming or engineering techniques to produce software, firmware, hardware, or any combination thereof. The term “article of manufacture” as used herein refers to code or logic implemented in hardware or computer readable media such as optical storage devices, and volatile or non-volatile memory devices. Such hardware may include, but is not limited to, field programmable gate arrays (FPGAs), application-specific integrated circuits (ASICs), complex programmable logic devices (CPLDs), programmable logic arrays (PLAs), microprocessors, or other similar processing devices. One or more implementations of the technology disclosed, or elements thereof can be implemented in the form of a computer product including a non-transitory computer readable storage medium with computer usable program code for performing the method steps indicated. Furthermore, one or more implementations of the technology disclosed, or elements thereof can be implemented in the form of an apparatus including a memory and at least one processor that is coupled to the memory and operative to perform exemplary method steps. Yet further, in another aspect, one or more implementations of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

### Terminology

As used herein, the term “sequenced data” refer to intensity data (e.g., intensity values) and non-intensity data. In some implementations, the segmentation and conditional base calling are performed on non-intensity data, such as on pH changes induced by the release of hydrogen ions during molecule extension. The pH changes are detected and converted to a voltage change that is proportional to the number of bases incorporated (e.g., in the case of Ion Torrent). Therefore, the sequence data disclosed herein includes voltage signals. In other implementations, the non-intensity data is constructed from nanopore sensing that uses biosensors to measure the disruption in current as an analyte passes through a nanopore or near its aperture while determining the identity of the base. For example, the Oxford Nanopore Technologies (ONT) sequencing is based on the following concept: pass a single strand of DNA (or RNA) through a membrane via a nanopore and apply a voltage difference across the membrane. The nucleotides present in the pore will affect the pore's electrical resistance, so current measurements over time can indicate the sequence of DNA bases passing through the pore. This electrical current signal (the ‘squiggle’ due to its appearance when plotted) is the raw data gathered by an ONT sequencer. These measurements are stored as 16-bit integer data acquisition (DAC) values, taken at e.g., 4 kHz frequency. With a DNA strand velocity of ˜450 base pairs per second, this gives approximately nine raw observations per base on average. This signal is then processed to identify breaks in the open pore signal corresponding to individual reads. These stretches of raw signal are base called—the process of converting DAC values into a sequence of DNA bases. In some implementations, the non-intensity data comprises normalized or scaled DAC values. Therefore, the sequence data disclosed herein can include current signals.

As used herein, the terms “polynucleotide” or “nucleic acids” refer to deoxyribonucleic acid (DNA), but where appropriate the skilled artisan will recognize that the systems and devices herein can also be utilized with ribonucleic acid (RNA). The terms should be understood to include, as equivalents, analogs of either DNA or RNA made from nucleotide analogs. The terms as used herein also encompasses cDNA, that is complementary, or copy, DNA produced from an RNA template, for example by the action of reverse transcriptase.

The single stranded polynucleotide molecules sequenced by the systems and devices herein can have originated in single-stranded form, as DNA or RNA or have originated in double-stranded DNA (dsDNA) form (e.g., genomic DNA fragments, PCR and amplification products and the like). Thus, a single stranded polynucleotide may be the sense or antisense strand of a polynucleotide duplex. Methods of preparation of single stranded polynucleotide molecules suitable for use in the method of the disclosure using standard techniques are well known in the art. The precise sequence of the primary polynucleotide molecules is generally not material to the disclosure, and may be known or unknown. The single stranded polynucleotide molecules can represent genomic DNA molecules (e.g., human genomic DNA) including both intron and exon sequences (coding sequence), as well as non-coding regulatory sequences such as promoter and enhancer sequences.

In some implementations, the nucleic acid to be sequenced through use of the current disclosure is immobilized upon a substrate (e.g., a substrate within a flow cell or one or more beads upon a substrate such as a flow cell, etc.). The term “immobilized” as used herein is intended to encompass direct or indirect, covalent or non-covalent attachment, unless indicated otherwise, either explicitly or by context. In some implementations covalent attachment may be preferred, but generally all that is required is that the molecules (e.g., nucleic acids) remain immobilized or attached to the support under conditions in which it is intended to use the support, for example in applications requiring nucleic acid sequencing.

As indicated above, the present disclosure comprises novel systems and devices for sequencing nucleic acids. As will be apparent to those of skill in the art, references herein to a particular nucleic acid sequence may, depending on the context, also refer to nucleic acid molecules which comprise such nucleic acid sequence. Sequencing of a target fragment means that a read of the chronological order of bases is established. The bases that are read do not need to be contiguous, although this is preferred, nor does every base on the entire fragment have to be sequenced during the sequencing. Sequencing can be carried out using any suitable sequencing technique, wherein nucleotides or oligonucleotides are added successively to a free 3′ hydroxyl group, resulting in synthesis of a polynucleotide chain in the 5′ to 3′ direction. The nature of the nucleotide added is preferably determined after each nucleotide addition. Sequencing techniques using sequencing by ligation, wherein not every contiguous base is sequenced, and techniques such as massively parallel signature sequencing (MPSS) where bases are removed from, rather than added to, the strands on the surface are also amenable to use with the systems and devices of the disclosure.

As described herein, the term “SBS” refers to sequencing-by-synthesis. In SBS, four fluorescently labeled modified nucleotides are used to sequence dense clusters of amplified DNA (possibly millions of clusters) present on the surface of a substrate (e.g., a flow cell). Various additional aspects regarding SBS procedures and methods, which can be utilized with the systems and devices herein, are disclosed in, for example, WO04018497, WO04018493 and U.S. Pat. No. 7,057,026 (nucleotides), WO05024010 and WO06120433 (polymerases), WO05065814 (surface attachment techniques), and WO 9844151, WO06064199 and WO07010251, the contents of each of which are incorporated herein by reference in their entirety.

As used herein, an element or step recited in the singular and proceeded with the word “a” or “an” should be understood as not excluding plural of said elements or steps, unless such exclusion is explicitly stated. Furthermore, references to “one implementation” are not intended to be interpreted as excluding the existence of additional implementations that also incorporate the recited features. Moreover, unless explicitly stated to the contrary, implementations “comprising” or “having” or “including” an element or a plurality of elements having a particular property may include additional elements whether or not they have that property.

In particular implementations, the reaction includes the incorporation of a fluorescently-labeled molecule to an analyte. The analyte may be an oligonucleotide and the fluorescently-labeled molecule may be a nucleotide. The desired reaction may be detected when an excitation light is directed toward the oligonucleotide having the labeled nucleotide, and the fluorophore emits a detectable fluorescent signal. In alternative implementations, the detected fluorescence is a result of chemiluminescence or bioluminescence. A desired reaction may also increase fluorescence (or Förster) resonance energy transfer (FRET), for example, by bringing a donor fluorophore in proximity to an acceptor fluorophore, decrease FRET by separating donor and acceptor fluorophores, increase fluorescence by separating a quencher from a fluorophore or decrease fluorescence by co-locating a quencher and fluorophore.

In some implementations, sensors (e.g., light detectors, photodiodes) are associated with corresponding pixel areas of a sample surface of a biosensor. As such, a pixel area is a geometrical construct that represents an area on the biosensor's sample surface for one sensor (or pixel). A sensor that is associated with a pixel area detects light emissions gathered from the associated pixel area when a desired reaction has occurred at a reaction site or a reaction chamber overlying the associated pixel area. In a flat surface implementation, the pixel areas can overlap. In some cases, a plurality of sensors may be associated with a single reaction site or a single reaction chamber. In other cases, a single sensor may be associated with a group of reaction sites or a group of reaction chambers.

As used herein, a “biosensor” includes a structure having a plurality of reaction sites and/or reaction chambers (or wells). A biosensor may include a solid-state imaging device (e.g., CCD or CMOS imager) and, optionally, a flow cell mounted thereto. The flow cell may include at least one flow channel that is in fluid communication with the reaction sites and/or the reaction chambers. As one specific example, the biosensor is configured to fluidically and electrically couple to a bioassay system. The bioassay system may deliver reactants to the reaction sites and/or the reaction chambers according to a predetermined protocol (e.g., sequencing-by-synthesis) and perform a plurality of imaging events. For example, the bioassay system may direct solutions to flow along the reaction sites and/or the reaction chambers. At least one of the solutions may include four types of nucleotides having the same or different fluorescent labels. The nucleotides may bind to corresponding oligonucleotides located at the reaction sites and/or the reaction chambers. The bioassay system may then illuminate the reaction sites and/or the reaction chambers using an excitation light source (e.g., solid-state light sources, such as light-emitting diodes or LEDs). The excitation light may have a predetermined wavelength or wavelengths, including a range of wavelengths. The excited fluorescent labels provide emission signals that may be captured by the sensors.

In alternative implementations, the biosensor may include electrodes or other types of sensors configured to detect other identifiable properties. For example, the sensors may be configured to detect a change in ion concentration. In another example, the sensors may be configured to detect the ion current flow across a membrane.

As used herein, a “cluster” is a colony of similar or identical molecules or nucleotide sequences or DNA strands. For example, a cluster can be an amplified oligonucleotide or any other group of a polynucleotide or polypeptide with a same or similar sequence. In other implementations, a cluster can be any element or group of elements that occupy a physical area on a sample surface. In implementations, clusters are immobilized to a reaction site and/or a reaction chamber during a base calling cycle.

As used herein, “base calling” identifies a nucleotide base in a nucleic acid sequence. Base calling refers to the process of determining a base call (A, C, G, T) for every cluster at a specific cycle. As an example, base calling can be performed utilizing four-channel, two-channel or one-channel methods and systems described in the incorporated materials of U.S. Patent Application Publication No. 2013/0079232. In particular implementations, a base calling cycle is referred to as a “sampling event.” In one dye and two-channel sequencing protocol, a sampling event comprises two illumination stages in time sequence, such that a pixel signal is generated at each stage. The first illumination stage induces illumination from a given cluster indicating nucleotide bases A and T in a AT pixel signal, and the second illumination stage induces illumination from a given cluster indicating nucleotide bases C and T in a CT pixel signal.

In some implementations, a computer-implemented method set forth herein can occur in real time while multiple images of an object are being obtained. Such real time analysis is particularly useful for nucleic acid sequencing applications wherein an array of nucleic acids is subjected to repeated cycles of fluidic and detection steps. Analysis of the sequencing data can often be computationally intensive such that it can be beneficial to perform the methods set forth herein in real time or in the background while other data acquisition or analysis algorithms are in process. Example real time analysis methods that can be used with the present methods are those used for the MiSeq, HiSeq, and NovaSeq sequencing devices commercially available from Illumina, Inc. (San Diego, Calif.) and/or described in US Pat. App. Pub. No. 2012/0020537 A1, which is incorporated herein by reference.

One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

The detailed description of some implementations will be better understood when read in conjunction with the appended drawings. To the extent that the figures illustrate diagrams of the functional blocks of various implementations, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be standalone programs, may be incorporated as subroutines in an operating system, may be functions in an installed software package, and the like. It should be understood that the various implementations are not limited to the arrangements and instrumentality shown in the drawings.

### CLAUSES

1. A system, comprising:
memory storing k-mer-specific centroids for k-mers, wherein the k-mer-specific centroids are learned by training a base calling pipeline to:
(i) represent base calls of an already base called sequence in k-mer-specific time series, wherein each of the k-mer-specific time series represents presence or absence of a particular k-mer at each sequencing cycle in a plurality of sequencing cycles across which the base calls are generated;
(ii) transform the k-mer-specific time series into predicted k-mer-specific centroids;
(iii) merge the predicted k-mer-specific centroids on a sequencing cycle-by-sequencing cycle basis to generate predicted per-sequencing cycle intensity values;
(iv) determine training loss (e.g., a transformation loss) based on comparing the predicted per-sequencing cycle intensity values against known intensity values of the base calls;
(v) update the predicted k-mer-specific centroids based on the determined transformation loss to generate updated k-mer-specific centroids; and
(vi) store the updated k-mer-specific centroids as the k-mer-specific centroids; and runtime logic configured to use the k-mer-specific centroids to base call bases in a yet-to-be base called sequence in dependence upon k-mer context.
2. The system of clause 1, wherein the k-mers are 4∧k permutations of k base positions, where 4 corresponds to four bases adenine (A), cytosine (C), guanine (G), and thymine (T).
3. The system of clause 2, wherein the k-mer-specific centroids are 4∧k k-mer-specific centroids.
4. The system of clause 1, wherein the base calls are discrete base calls.
5. The system of clause 4, wherein the discrete base calls are encoded as binary permutations across two channels.
6. The system of clause 1, wherein the k-mer-specific centroids are learned by iteratively training the base calling pipeline to iteratively generate the updated k-mer-specific centroids for base calls of a plurality of already base called sequences.
7. The system of clause 1, wherein the k-mer-specific centroids correct for chemistry modulation effects.
8. The system of clause 7, wherein the k-mer-specific centroids correct for k-mer dependent effects.
9. The system of clause 7, wherein the k-mer-specific centroids correct for fully functional nucleoside triphosphate (FFN) modulation effects.
10. The system of clause 7, wherein the k-mer-specific centroids correct for quenching effects.
11. The system of clause 1, wherein the k-mer-specific time series are transformed into the predicted k-mer-specific centroids using k-mer-specific convolution kernels.
12. The system of clause 11, wherein the k-mer-specific convolution kernels are initialized as identity matrices.
13. The system of clause 1, wherein the k-mer-specific time series are transformed into the predicted k-mer-specific centroids using backpropagation.
14. The system of clause 13, wherein the backpropagation is implemented by an Adam optimizer.
15. The system of clause 1, wherein each of the predicted k-mer-specific centroids (or k-mer-specific time series) is corrected for phasing effect to generate a corrected k-mer-specific centroid (or k-mer-specific time series).
16. The system of clause 15, wherein corrected k-mer-specific centroids are merged on a sequencing cycle-by-sequencing cycle basis to generate the predicted per-sequencing cycle intensity values.
17. A computer-implemented method of base calling a target cluster, comprising:
accessing current intensity data for a current sequencing cycle of a sequencing run and context intensity data for at least one of a preceding sequencing cycle or a succeeding sequencing cycle;
identifying a base context of the target cluster based on the context intensity data;
accessing a plurality of k-mer-specific centroids for k-mers to determine at least one k-mer-specific centroid corresponding to the base context of the target cluster,
wherein each of the plurality of k-mer-specific centroids represents a mean value of intensity of clusters with a particular k-mer-specific base context, and
wherein the plurality of k-mer-specific centroids is learned by training a base calling pipeline to process as input base calls of an already base called sequence in k-mer-specific time series and output the plurality of k-mer-specific centroids, each of the k-mer-specific time series representing presence or absence of a particular k-mer at each sequencing cycle in a plurality of sequencing cycles across which the base calls are generated; and
base calling the target cluster by comparing the current intensity data with the at least one k-mer-specific centroid.
18. The method of clause 17, wherein the k-mers are 4∧k permutations of k base positions, wherein 4 corresponds to four bases adenine (A), cytosine (C), guanine (G), and thymine (T).
19. The method of clause 18, wherein the k-mer-specific centroids are 4∧k k-mer-specific centroids.
20 The method of clause 17, wherein the base calls are discrete base calls.
21. The method of clause 20, wherein the discrete base calls are encoded as binary permutations across two channels.
22. The method of clause 17, wherein the k-mer-specific centroids are learned by iteratively training the base calling pipeline to iteratively generate updated k-mer-specific centroids for base calls of a plurality of already base called sequences.
23. The method of clause 17, wherein the k-mer-specific centroids correct for chemistry modulation effects.
24 The method of clause 23, wherein the k-mer-specific centroids correct for k-mer dependent effects.
25. The method of clause 23, wherein the k-mer-specific centroids correct for fully functional nucleoside triphosphate (FFN) modulation effects.
26. The method of clause 23, wherein the k-mer-specific centroids correct for quenching effects.
27. The method of clause 17, wherein the k-mer-specific time series are transformed into the k-mer-specific centroids using k-mer-specific convolution kernels.
28. The method of clause 27, wherein the k-mer-specific convolution kernels are initialized as identity matrices.
29. The method of clause 17, wherein the k-mer-specific time series are transformed into the k-mer-specific centroids using backpropagation.
30. The method of clause 29, wherein the backpropagation is implemented by an Adam optimizer.
31. The method of clause 29, wherein the base calling pipeline merges the k-mer-specific centroids on a sequencing cycle-by-sequencing cycle basis to generate predicted per-sequencing cycle intensity values, determines a training loss (e.g., a transformation loss) by comparing the predicted per-sequencing cycle intensity values against known intensity values of the base calls, and updates the k-mer-specific centroids based on the training loss.
32. The method of clause 31, wherein each of the k-mer-specific centroids is further corrected for phasing effect to generate a corrected k-mer-specific centroid.
33. The method of clause 32, wherein corrected k-mer-specific centroids are merged on the sequencing cycle-by-sequencing cycle basis to generate the predicted per-sequencing cycle intensity values.
34. A computer-implemented method of training a base calling pipeline, comprising:
receiving as training samples base calls of an already base called sequence in k-mer-specific time series, wherein each of the k-mer-specific time series represents presence or absence of a particular k-mer at each sequencing cycle in a plurality of sequencing cycles across which the base calls are generated;
transforming the k-mer-specific time series into k-mer-specific centroids for k-mers;
merging the k-mer-specific centroids on a sequencing cycle-by-sequencing cycle basis to generate predicted per-sequencing cycle intensity values;
determining a training loss (e.g., a transformation loss) based on comparing the predicted per-sequencing cycle intensity values against known intensity values of the base calls; and
updating the k-mer-specific centroids based on the determined training loss to generate updated k-mer-specific centroids.
35. The method of clause 34, wherein the k-mers are 4∧k permutations of k base positions, where 4 corresponds to four bases adenine (A), cytosine (C), guanine (G), and thymine (T).
36. The method of clause 35, wherein the k-mer-specific centroids and the updated k-mer-specific centroids are 4∧k k-mer-specific centroids, respectively.
37 The method of clause 34, wherein the base calls are discrete base calls.
38. The method of clause 37, wherein the discrete base calls are encoded as binary permutations across two channels.
39. The method of clause 34, wherein the k-mer-specific centroids are learned by iteratively generating the updated k-mer-specific centroids for base calls of a plurality of already base called sequences.
40. The method of clause 34, wherein the k-mer-specific centroids correct for chemistry modulation effects.
41. The method of clause 40, wherein the k-mer-specific centroids correct for k-mer dependent effects.
42 The method of clause 40, wherein the k-mer-specific centroids correct for fully functional nucleoside triphosphate (FFN) modulation effects.
43. The method of clause 40, wherein the k-mer-specific centroids correct for quenching effects.
44 The method of clause 34, wherein the k-mer-specific time series are transformed into the k-mer-specific centroids using k-mer-specific convolution kernels.
45. The method of clause 44 wherein the k-mer-specific convolution kernels are initialized as identity matrices.
46. The method of clause 34, wherein the k-mer-specific time series are transformed into the predicted k-mer-specific centroids using backpropagation.
47. The method of clause 46, wherein the backpropagation is implemented by an Adam optimizer.
48. A non-transitory computer readable storage medium impressed with computer program instructions to base call a target cluster, the instructions, when executed on a processor, implement a method comprising:
accessing current intensity data for a current sequencing cycle of a sequencing run and context intensity data for at least one of a preceding sequencing cycle or a succeeding sequencing cycle;
identifying a base context of the target cluster based on the context intensity data;
accessing a plurality of k-mer-specific centroids for k-mers to determine at least one k-mer-specific centroid corresponding to the base context of the target cluster,
wherein each of the plurality of k-mer-specific centroids represents a mean value of intensity of clusters with a particular k-mer-specific base context, and
wherein the plurality of k-mer-specific centroids is learned by training a base calling pipeline to process as input base calls of an already base called sequence in k-mer-specific time series and output the plurality of k-mer-specific centroids, each of the k-mer-specific time series representing presence or absence of a particular k-mer at each sequencing cycle in a plurality of sequencing cycles across which the base calls are generated; and
base calling the target cluster by comparing the current intensity data with the at least one k-mer-specific centroid.
49. The non-transitory computer readable storage medium of clause 48, wherein the k-mers are 4∧k permutations of k base positions, wherein 4 corresponds to four bases adenine (A), cytosine (C), guanine (G), and thymine (T).
50. The non-transitory computer readable storage medium of clause 49, wherein the k-mer-specific centroids are 4∧k k-mer-specific centroids.
51. The non-transitory computer readable storage medium of clause 48, wherein the base calls are discrete base calls.
52. The non-transitory computer readable storage medium of clause 51, wherein the discrete base calls are encoded as binary permutations across two channels.
53. The non-transitory computer readable storage medium of clause 48, wherein the k-mer-specific centroids are learned by iteratively training the base calling pipeline to iteratively generate updated k-mer-specific centroids for base calls of a plurality of already base called sequences.
54. The non-transitory computer readable storage medium of clause 48, wherein the k-mer-specific centroids correct for chemistry modulation effects.
55. The non-transitory computer readable storage medium of clause 54, wherein the k-mer-specific centroids correct for k-mer dependent effects.
56. The non-transitory computer readable storage medium of clause 54, wherein the k-mer-specific centroids correct for fully functional nucleoside triphosphate (FFN) modulation effects.
57. The non-transitory computer readable storage medium of clause 54, wherein the k-mer-specific centroids correct for quenching effects.
58. The non-transitory computer readable storage medium of clause 48, wherein the k-mer-specific time series are transformed into the k-mer-specific centroids using k-mer-specific convolution kernels.
59. The non-transitory computer readable storage medium of clause 58, wherein the k-mer-specific convolution kernels are initialized as identity matrices.
60. The non-transitory computer readable storage medium of clause 48, wherein the k-mer-specific time series are transformed into transformed k-mer-specific time series using backpropagation.
61. The non-transitory computer readable storage medium of clause 60, wherein the backpropagation is implemented by an Adam optimizer.
62. The non-transitory computer readable storage medium of clause 60, wherein the base calling pipeline merges the transformed k-mer-specific time series on a sequencing cycle-by-sequencing cycle basis to generate predicted per-sequencing cycle intensity values, determines a training loss (e.g., a transformation loss) by comparing the predicted per-sequencing cycle intensity values against known intensity values of the base calls, and updates the k-mer-specific centroids based on the training loss.
63. The non-transitory computer readable storage medium of clause 62, wherein each of the transformed k-mer-specific time series is further corrected for phasing effect to generate a corrected k-mer-specific time series.
64. The non-transitory computer readable storage medium of clause 63, wherein corrected k-mer-specific time series are merged on the sequencing cycle-by-sequencing cycle basis to generate the predicted per-sequencing cycle intensity values.
65. A non-transitory computer readable storage medium impressed with computer program instructions to train a base calling pipeline, the instructions, when executed on a processor, implement a method comprising:
receiving as training samples base calls of an already base called sequence in k-mer-specific time series, wherein each of the k-mer-specific time series represents presence or absence of a particular k-mer at each sequencing cycle in a plurality of sequencing cycles across which the base calls are generated;
transforming the k-mer-specific time series into k-mer-specific centroids for k-mers;
merging the k-mer-specific centroids on a sequencing cycle-by-sequencing cycle basis to generate predicted per-sequencing cycle intensity values;
determining a training loss (e.g., a transformation loss) based on comparing the predicted per-sequencing cycle intensity values against known intensity values of the base calls; and
updating the k-mer-specific centroids based on the determined training loss to generate updated k-mer-specific centroids.
66. The non-transitory computer readable storage medium of clause 65, wherein the k-mers are 4∧k permutations of k base positions, where 4 corresponds to four bases adenine (A), cytosine (C), guanine (G), and thymine (T).
67. The non-transitory computer readable storage medium of clause 66, wherein the k-mer-specific centroids and the updated k-mer-specific centroids are 4∧k k-mer-specific centroids, respectively.
68. The non-transitory computer readable storage medium of clause 65, wherein the base calls are discrete base calls.
69 The non-transitory computer readable storage medium of clause 68, wherein the discrete base calls are encoded as binary permutations across two channels.
70 The non-transitory computer readable storage medium of clause 65, wherein the k-mer-specific centroids are learned by iteratively generating the updated k-mer-specific centroids for base calls of a plurality of already base called sequences.
71. The non-transitory computer readable storage medium of clause 65, wherein the k-mer-specific centroids correct for chemistry modulation effects.
72. The non-transitory computer readable storage medium of clause 71, wherein the k-mer-specific centroids correct for k-mer dependent effects.
73. The non-transitory computer readable storage medium of clause 71, wherein the k-mer-specific centroids correct for fully functional nucleoside triphosphate (FFN) modulation effects.
74. The non-transitory computer readable storage medium of clause 71, wherein the k-mer-specific centroids correct for quenching effects.
75. The non-transitory computer readable storage medium of clause 65, wherein the k-mer-specific time series are transformed into the k-mer-specific centroids using k-mer-specific convolution kernels.
76. The non-transitory computer readable storage medium of clause 75, wherein the k-mer-specific convolution kernels are initialized as identity matrices.
77. The non-transitory computer readable storage medium of clause 65, wherein the k-mer-specific time series are transformed into the predicted k-mer-specific centroids using backpropagation.
78. The non-transitory computer readable storage medium of clause 77, wherein the backpropagation is implemented by an Adam optimizer.
79. The non-transitory computer readable storage medium of clause 65, wherein each of the k-mer-specific centroids for k-mers is further corrected for phasing effect to generate a corrected k-mer-specific centroid.
80. The non-transitory computer readable storage medium of clause 79, wherein corrected k-mer-specific centroids are merged on the sequencing cycle-by-sequencing cycle basis to generate the predicted per-sequencing cycle intensity values.
81. A system, comprising:
memory storing k-mer-specific centroids for k-mers that are sequences of k bases, wherein the k-mer-specific centroids are learned by training a base calling pipeline to:
represent a k-mer as a target base and as one of 4∧k permutations of bases determined by the target base and adjoining bases in a sequence of k bases;
represent at least the target base in the k-mer as one or more categorical intensity values, with at least one categorical value per intensity collection channel;
apply a transformation of the k-mer into a predicted centroid of one or more real valued intensity values expected to be collected for the target base given the k-mer;
determine a training loss (e.g., a transformation loss) by comparing the predicted centroid of real valued intensity values with one or more intensity values actually collected during sequencing for the target base;
update the transformation from the k-mer to the predicted centroid;
after learning the transformation, store predicted centroids of collected intensity values for target bases for each of the 4∧k permutations; and
runtime logic configured to use the k-mer-specific centroids to base call bases in a sequence in dependence upon k-mer context.
82. The system of clause 81, wherein there are two intensity collection channels, further including training the base calling pipeline to:
represent the target base as two binary categorical intensity values; and predict a centroid for the target base as two real valued intensity values.
83. The system of clause 82, further including training the base calling pipeline to:
produce a 2×2 linear transform matrix or a 3×3 affine transform matrix specific to each k-mer permutation; and apply the 2×2 linear transform matrix or the 3×3 affine transform matrix as the transformation.
84. The system of clause 81, wherein there are two intensity collection channels, further including training the base calling pipeline to:
produce a matrix of coefficients as the transformation, wherein the coefficients directly convert the one or more categorical intensity values of the target base and the context k-mer into two real valued intensity values; and after the learning, enumerate the permutations of the k-mers to predict the centroids of collected intensity values for target bases for each of the 4∧k permutations.
85. The system of clause 81, wherein the transformation takes into account context-dependent phasing effects.
86. The system of clause 81, wherein the predicted centroids are further adjusted after application of the transformation to take into account intensity effects of one or more of fully functional nucleoside triphosphate (FFN) incorporation modulation effects, quantum yield and quenching effects.
87. The system of clause 81, wherein the predicted centroids are further adjusted after application of the transformation to take into account collected intensity effects of background, signal generation decay relative to a current cycle of base calling, scale of a particular cluster being base called, camera gain, or ramping of laser power applied to generate intensity signals.
88. The system of clause 81, wherein the update to the transformation is trained using backpropagation.
89 The system of clause 88, wherein the backpropagation is implemented by an Adam optimizer.
90. The system of clause 81, wherein the k-mer has a length k=5 and 1024 centroids are predicted for 1024 permutations of k-mers.
91. The system of clause 81, wherein bases that appear in the k-mer are adenine (A), cytosine (C), guanine (G), and thymine (T).
92. A trained base calling production system, comprising:
an input forming module that
   represents a k-mer to be called as a target base and as one of 4∧(k−1) permutations of bases determined by bases adjoining the target base in the k-mer to be called; and
   represents at least the target base in the k-mer to be called as one or more real intensity values, with at least one real value per intensity collection channel;
a centroid access module that determines alternative predicted centroids, based on a context of the bases adjoining the target base, the determined alternative predicted centroids corresponding to alternative values of the target base in the k-mer to be called; and
a prediction module that compares the alternative predicted centroids to the real intensity values collected for the k-mer to be called and determines the target base using the alternative predicted centroids that is a shortest distance from the real intensity values collected.
93. The system of clause 92, wherein the system includes memory storing k-mer-specific centroids for k-mers that are sequences of k bases, wherein the k-mer-specific centroids are learned by training a base calling pipeline to:
represent a k-mer as a target base and as one of 4∧k permutations of bases determined by the target base and adjoining bases in a sequence of k bases;
represent at least the target base in the k-mer as one or more categorical intensity values, with at least one categorical value per intensity collection channel;
apply a transformation of the k-mer into a predicted centroid of one or more real valued intensity values expected to be collected for the target base given the k-mer;
determine a training loss (e.g., a transformation loss) by comparing the predicted centroid of real valued intensity values with one or more intensity values actually collected during sequencing for the target base; and
update the transformation from the k-mer to the predicted centroid.
94. The system of clause 92, wherein there are two intensity collection channels, further including:
the input forming module represents the target base as two binary categorical intensity values; and
the prediction module predicts the alternative predicted centroid for the target base as having two real valued intensity values.
95. The system of clause 93, further including training the base calling pipeline to:
produce a 2×2 transform matrix or a 3×3 transform matrix specific to each k-mer permutation; and
apply the 2×2 transform matrix or the 3×3 transform matrix as the transformation.
96. The system of clause 93, wherein there are two intensity collection channels, further including training the base calling pipeline to:
produce a matrix of coefficients as the transformation, wherein the coefficients directly convert the one or more categorical intensity values of the target base and the context the k-mer into two real valued intensity values; and
after the learning, enumerate the permutations of the k-mers to predict the centroids of collected intensity values for target bases for each of the 4∧k permutations.
97. The system of clause 92, wherein the prediction module further adjusts the predicted centroids before proceeding to determine the target bases context dependent phasing effects.
98. The system of clause 92, wherein the prediction module further adjusts the predicted centroids before proceeding to determine the target bases to take into account intensity effect of one or more of fully functional nucleoside triphosphate (FFN) incorporation modulation effects, quantum yield and quenching effects.
99. The system of clause 93, wherein the update to the transformation is trained using backpropagation.
100. The system of clause 99, wherein the backpropagation is implemented by an Adam optimizer.
101. The system of clause 92, wherein the k-mer has a length k=5 and 1024 centroids are predicted for 1024 permutations of k-mers.
102. The system of clause 92, wherein bases that appear in the k-mer are adenine (A), cytosine (C), guanine (G), and thymine (T).

While the present invention is disclosed by reference to the preferred implementations and examples detailed above, it is to be understood that these examples are intended in an illustrative rather than in a limiting sense. It is contemplated that modifications and combinations will readily occur to those skilled in the art, which modifications and combinations will be within the spirit of the invention and the scope of the following claims.

What is claimed is:

1. A computer-implemented method of base calling a target cluster, comprising:
accessing current intensity data for a current sequencing cycle of a sequencing run and context intensity data for at least one of a preceding sequencing cycle or a succeeding sequencing cycle;
identifying a base context of the target cluster based on the context intensity data;
accessing a plurality of k-mer-specific centroids for k-mers to determine at least one k-mer-specific centroid corresponding to the base context of the target cluster,
wherein each of the plurality of k-mer-specific centroids represents a mean value of intensities of clusters with a particular k-mer-specific base context, and
wherein the plurality of k-mer-specific centroids are accessed using a base calling pipeline to process, as input, base calls of an already base called sequence in 4∧k permutations of k-mer-specific time series and provide, as output, the plurality of k-mer-specific centroids, each of the 4∧k permutations of k-mer-specific time series representing presence or absence of a particular k-mer at each sequencing cycle in a plurality of sequencing cycles across which the base calls are generated; and
base calling the target cluster by comparing the current intensity data with the at least one k-mer-specific centroid.

2. The computer-implemented method of claim 1, wherein the k-mers are 4∧k permutations of k base positions, wherein 4 corresponds to four bases adenine (A), cytosine (C), guanine (G), and thymine (T).

3. The computer-implemented method of claim 2, wherein the k-mer-specific centroids are 4∧k k-mer-specific centroids.

4. The computer-implemented method of claim 1, wherein the base calls are discrete base calls.

5. The computer-implemented method of claim 4, wherein the discrete base calls are encoded as binary permutations across two channels.

6. The computer-implemented method of claim 1, wherein the k-mer-specific centroids are learned by iteratively training the base calling pipeline to iteratively generate updated k-mer-specific centroids for base calls of a plurality of already base called sequences.

7. The computer-implemented method of claim 1, wherein the k-mer-specific centroids correct for chemistry modulation effects.

8. The computer-implemented method of claim 7, wherein the k-mer-specific centroids correct for k-mer dependent effects.

9. The computer-implemented method of claim 7, wherein the k-mer-specific centroids correct for fully functional nucleoside triphosphate (FFN) modulation effects.

10. The computer-implemented method of claim 7, wherein the k-mer-specific centroids correct for quenching effects.

11. The computer-implemented method of claim 1, wherein the k-mer-specific time series are transformed into the k-mer-specific centroids using k-mer-specific convolution kernels.

12. The computer-implemented method of claim 11, wherein the k-mer-specific convolution kernels are initialized as identity matrices.

13. The computer-implemented method of claim 1, wherein the k-mer-specific time series are transformed into the k-mer-specific centroids using backpropagation.

14. The computer-implemented method of claim 13, wherein the backpropagation is implemented by an Adam optimizer.

15. The computer-implemented method of claim 13, wherein the base calling pipeline
merges the k-mer-specific centroids on a sequencing cycle-by-sequencing cycle basis to generate predicted per-sequencing cycle intensity values,
determines a training loss by comparing the predicted per-sequencing cycle intensity values against known intensity values of the base calls, and
updates the k-mer-specific centroids based on the training loss.

16. The computer-implemented method of claim 15, wherein each of the k-mer-specific centroids is further corrected for phasing effect to generate a corrected k-mer-specific centroid.

17. The computer-implemented method of claim 16, wherein corrected k-mer-specific centroids are merged on the sequencing cycle-by-sequencing cycle basis to generate the predicted per-sequencing cycle intensity values.

18. A non-transitory computer readable storage medium comprising computer program instructions that, when executed on a processor, implement a method comprising:
accessing current intensity data for a current sequencing cycle of a sequencing run and context intensity data for at least one of a preceding sequencing cycle or a succeeding sequencing cycle;
identifying a base context of a target cluster based on the context intensity data;
accessing a plurality of k-mer-specific centroids for k-mers to determine at least one k-mer specific centroid corresponding to the base context of the target cluster,
wherein each of the plurality of k-mer-specific centroids represents a mean value of intensities of clusters with a particular k-mer-specific base context, and
wherein the plurality of k-mer-specific centroids are accessed using a base calling pipeline to process, as input, base calls of an already base called sequence in 4∧k permutations of k-mer-specific time series and provide, as output, the plurality of k-mer-specific centroids, each of the k-mer-specific time series representing presence or absence of a particular k-mer at each sequencing cycle in a plurality of sequencing cycles across which the base calls are generated; and
base calling the target cluster by comparing the current intensity data with the at least one k-mer-specific centroid.

19. A system comprising:
at least one processor; and
computer program instructions that, when executed on the at least one processor, cause the system to:
access current intensity data for a current sequencing cycle of a sequencing run and context intensity data for at least one of a preceding sequencing cycle or a succeeding sequencing cycle;
identify a base context of a target cluster based on the context intensity data;
access a plurality of k-mer-specific centroids for k-mers to determine at least one k-mer-specific centroid corresponding to the base context of the target cluster,
wherein each of the plurality of k-mer-specific centroids represents a mean value of intensities of clusters with a particular k-mer-specific base context, and
wherein the plurality of k-mer-specific centroids are accessed using a base calling pipeline to process, as input, base calls of an already base called sequence in 4∧k permutations of k-mer-specific time series and provide, as output, the plurality of k-mer-specific centroids, each of the k-mer-specific time series representing presence or absence of a particular k-mer at each sequencing cycle in a plurality of sequencing cycles across which the base calls are generated; and
base call the target cluster by comparing the current intensity data with the at least one k-mer-specific centroid.

20. The system of claim 19, wherein the k-mers are 4∧k permutations of k base positions, wherein 4 corresponds to four bases adenine (A), cytosine (C), guanine (G), and thymine (T).
